(19) United States
(12) Patent Application Publication
(10) Pub. No.: US 2025/0054079 A1
(43) Pub. Date: Feb. 13, 2025

(54) BALANCING OF ENVIRONMENTAL ATTRIBUTES IN PRODUCTION NETWORKS

(71) Applicant: BASF SE, Ludwigshafen am Rhein (DE)

(72) Inventors: Bodo FREY, Ludwigshafen am Rhein (DE); Marion MAYER, Ludwigshafen am Rhein (DE); Mathias FEYEN, Ludwigshafen am Rhein (DE); Jasmin BECKENBACH, Ludwigshafen am Rhein (DE); Ana ALBA PEREZ, Ludwigshafen am Rhein (DE)

(21) Appl. No.: 18/720,684

(22) PCT Filed: Dec. 19, 2022
(86) PCT No.: PCT/EP2022/086749

§ 371 Date: Jun. 17, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G06Q 50/04; G06Q 10/30

(52) U.S. Cl. CPC G06Q 50/04; G06Q 10/30

(57) ABSTRACT

Disclosed are methods, apparatuses, and systems for producing at least one chemical product associated with one or more environmental attribute(s) and for assigning at least one environmental attribute to at least one chemical product produced by a chemical production network.

### TECHNICAL FIELD

The present disclosure relates to methods, apparatuses and systems for producing at least one chemical product associated with one or more environmental attribute(s) and for assigning at least one environmental attribute to at least one chemical product produced by a chemical production network.

### TECHNICAL BACKGROUND

In supply chains the environmental impact of each supply chain participants is of great interest. Transparency between the participants can aid collective reduction of environmental impacts to combat climate change. However, data sharing of environmental impact data is hindered by the lack of common data standards and the lack of trusted data platforms. In addition, the highly specific and centralized setup of data systems today makes exchange and sharing for collective action laborious. Hence, there is a need to develop metrices quantifying the environmental impact of produced products, to simplify data standards relating to environmental impact and to broadly enable a secure exchange of supply chain data relating to the environmental impact.

### SUMMARY OF THE INVENTION

In one aspect disclosed is a method for producing at least one chemical product associated with one or more environmental attribute(s), wherein the chemical product is produced by a chemical production network, the method comprising:
providing one or more input material(s) associated with one or more environmental attribute(s) to the chemical production network, providing material data related to the one or more input material(s) and respective environmental attributes to a computing interface,
allocating the one or more environmental attribute(s) to a virtual balancing system, wherein the virtual balancing system includes balancing accounts associated with one or more environmental attribute(s),
producing the at least one chemical product,
assigning the at least one environmental attribute from at least one balancing account associated with the respective environmental attribute to the at least one chemical product.

In another aspect disclosed is a computer-implemented method for assigning at least one environmental attribute to at least one chemical product produced by a chemical production network, the method comprising:
providing material data related to the one or more input material(s) and respective environmental attributes to a computing interface,
allocating the one or more environmental attribute(s) to a virtual balancing system, wherein the virtual balancing system includes balancing accounts associated with one or more environmental attribute(s),
assigning the at least one environmental attribute from at least one balancing account associated with the respective environmental attribute to the at least one chemical product.

In another aspect disclosed is a computer-implemented method for assigning at least one environmental attribute to at least one chemical product produced by a chemical production network, the method comprising:
providing access to a virtual balancing system, wherein the virtual balancing system includes more than one balancing account associated with at least one environmental attribute and an environmental attribute balance with environmental attribute(s) allocated from one or more input material(s) provided to the chemical production network,
based on or more environmental attribute(s) to be assigned to the at least one chemical product selecting at least one balancing account from the virtual balancing system, assigning the at least one environmental attribute from the at least one balancing account associated with the respective environmental attribute to the at least one chemical product.

In another aspect disclosed is an apparatus for assigning at least one environmental attribute to at least one chemical product produced by a chemical production network, the method comprising:
a material data provider configured to provide material data related to the one or more input material(s) and respective environmental attributes to a computing interface,
an inbound allocator configured to allocate the one or more environmental attribute(s) to a virtual balancing system, wherein the virtual balancing system includes balancing accounts associated with one or more environmental attribute(s),
an outbound assignor configured to assign the at least one environmental attribute from at least one balancing account associated with the respective environmental attribute to the at least one chemical product.

In another aspect disclosed is an apparatus for assigning at least one environmental attribute to at least one chemical product produced by a chemical production network, the method comprising:
an account provider configured to provide access to a virtual balancing system, wherein the virtual balancing system includes more than one balancing account associated with at least one environmental attribute and an environmental attribute balance with environmental attribute(s) allocated from one or more input material(s) provided to the chemical production network,
an outbound assignor configured to select at least one balancing account from the virtual balancing system based on or more environmental attribute(s) to be assigned to the at least one chemical product and configured to assign the at least one environmental attribute from the at least one balancing account associated with the respective environmental attribute to the at least one chemical product.

In another aspect disclosed is a system for producing at least one chemical product associated with one or more environmental attributes, wherein the chemical product is produced by a chemical production network including one or more production chain(s), the system comprising:
a chemical production network configured to produce at least one chemical product based on one or more input material(s) provided to the chemical production network, wherein the one or more input material(s) are associated with one or more environmental attribute(s);
an inbound allocator configured to provide material data related to the one or more input material(s) and respective environmental attributes to a computing interface and to allocate the one or more environmental attribute(s) to a virtual balancing system, wherein the virtual balancing system includes balancing accounts associated with one or more environmental attribute(s),
an outbound assignor configured to assign the at least one environmental attribute from the at least one balancing account associated with the respective environmental attribute to the at least one chemical product.

In yet another aspect disclosed is a computer element, in particular a computer program product or a computer readable medium, with instructions, which when executed by one or more processor(s) are configured to carry out the steps of any of the methods disclosed herein. In yet another aspect disclosed is a computer element, in particular a computer program product or a computer readable medium, with instructions, which when executed by one or more processor(s) cause any of the apparatuses disclosed herein to perform any of the methods disclosed herein.

Disclosed is in yet another aspect the use of the chemical product associated with one or more environmental attribute(s) as provided by any of the methods disclosed herein and/or produced by a chemical production network as provided by any of the methods disclosed herein to produce at least one discrete product or at least one end product of a product supply chain associated with the one or more environmental attribute(s). Disclosed is in yet another aspect a method for producing at least one discrete product or at least one end product of a product supply chain associated with the one or more environmental attribute(s), wherein the chemical product associated with one or more environmental attribute(s) as provided by any of the methods disclosed herein and/or produced by a chemical production network as provided by any of the methods disclosed herein is provided and/or used to produce the at least one discrete product or at least one end product of a product supply chain associated with the one or more environmental attribute(s).

Disclosed is in yet another aspect the use of the one or more environmental attribute(s) assigned to the chemical product identifier as provided by any of the methods disclosed herein to provide at least one discrete product identifier relating at least one discrete product or at least one end product identifier relating at least one end product of a product supply chain to the one or more environmental attribute(s). Disclosed is in yet another aspect a method for providing at least one discrete product identifier relating at least one discrete product or at least one end product identifier relating at least one end product of a product supply chain to the one or more environmental attribute(s), wherein the one or more environmental attribute(s) assigned to the chemical product identifier as provided by any of the methods disclosed herein are provided and/or used to provide the at least one discrete product identifier relating at least one discrete product or at least one end product identifier relating at least one end product of a product supply chain to the one or more environmental attribute(s).

### EMBODIMENTS

Any disclosure and embodiments described herein relate to the methods, the apparatuses, the computer elements and uses lined out above or below and vice versa. The benefits provided by any of the embodiments and examples equally apply to all other embodiments and examples and vice versa.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

The methods, apparatuses, systems, and computer elements disclosed herein provide an efficient way to track environmental attributes in chemical processing and provide chemical products with positive environmental impact through the value chain. By using a virtual accounting system for balancing environmental attributes associated with input materials, such attributes can be efficiently assigned to chemical products produced in chemical productions networks. Specifically for chemical networks that produce more than one chemical product from more than one input material via interconnected, connected and non-connected production chains, the use of the virtual accounting system allows to reliably assign environmental attributes in line with the physical setup of the chemical production network and to tailor the digital assets associated with the chemical product to the needs of customers. The virtual accounting system and the associated metadata structure further allows to decouple the complexity in material flow of chemical production networks while still allowing to tailor environmental impact to each chemical product. This way the environmental impact of the produced chemical product can be determined in line with the physical set up of the chemical production network and the tailored needs of customers. Moreover, the environmental property of the chemical products produced by the chemical production network can be made transparent to customers further processing the chemical products. By providing chemical product identifiers associated with at least one environmental attribute, the environmental attributes and as such the digital asset attached to the chemical product can be adjusted to customer needs.

The environmental attribute may be a digital asset associated with the input material or chemical product. The environmental attribute may digitally specify the environmental impact of the input material or the chemical product. The environmental attribute may relate to a renewable, a bio-based and/or a recycled content e.g., of the input material and/or chemical product. The environmental attribute may include a qualitative data point relating to the type of impact e.g., in view of the input material or the chemical product. The environmental attribute may specify a type such as recycled, renewable and/or bio-based. The qualitative data point may be converted to a quantitative measure such as balancing units. The environmental attribute may include a quantitate data point relating to the type of impact e.g., in view of the input material or the chemical product recycled content, renewable content or bio-based content. The environmental attribute may specify recycled, renewable and/or bio-based content.

Environmental attribute(s) may refer to any property related to the environmental impact. Such property may be a property of input material(s) or chemical product(s). The environmental attribute may indicate an environmental performance of input material(s), the chemical production network and/or chemical product(s). The environmental attribute may be derived from properties of the input material(s), the chemical production network and/or the chemical product(s). The environmental attribute may be associated with the environmental impact of one or more material(s) at any stage during their lifecycle. The stages of the material or product lifecycle may include the stages of providing raw material, producing products, such as intermediate products or end products, using products, treating end-of-life products, recycling end-of-life products, disposing end-of-life products, reusing components from end-of-life products or any subset of stages. The environmental attribute may be tracked through any activity of one or more entities participating at any stage of the lifecycle of one or more material(s) or product(s). Environmental attributes associated with any activity of one or more entities participating at any stage of the lifecycle of one or more material(s) or product(s) may be accumulated or aggregated.

The environmental attribute may include one or more characteristic(s) that are attributable to environmental impact of the input material(s), chemical product(s), intermediate product(s) and/or end product(s). The environmental attribute may include environmental, technical, recyclability or circularity characteristics(s) associated with the environmental impact of the input material(s), chemical product(s), intermediate product(s) and/or end product(s).

Environmental characteristic(s) may specify or quantify ecological criteria associated with the environmental impact of input material(s), chemical product(s), intermediate product(s) and/or end product(s). Environmental characteristic(s) may for example include recycled content, bio-based content, renewable content or the like.

Technical characteristic(s) may specify or quantify product performance at least indirectly associated with the environmental impact. Technical characteristic(s) may for example include product composition data, bill of materials, product specification data, product component data, product safety data, application property data, application instructions or product quality data.

Chemical production networks may include multiple types of production processes for producing different chemical products from input materials. The chemical production network may include a complex production network producing multiple chemical products in multiple production or value chains. A production or value chain may include one or more process(es) configured to produce one chemical product or chemical product class from one or more input material(s). The chemical production network may include connected, interconnected and/or non-connected production chains. The chemical production network may produce from input materials multiple intermediates and from intermediates chemical products. Input material may enter the chemical production network at entry points. Chemical product may leave the production network at exit points.

The chemical production network may comprise one or more entry points at which input materials are provided to the chemical production network. Input material may include fossil material, non-fossil material or both. Fossil input material may include crude oil, natural gas or coal. Non fossil input material may include renewable material, bio-based material or recycled materials. Input material may include feedstock for a gasification plant, a steam cracker or synthesis gas plant. Input material may include synthesis gas produced from fossil feedstock, non-fossil feedstock or both. Input material may include for example pyrolysis oil from recycled waste, syngas produced from recycled waste, naphtha produced from bio-based material (bio-naphtha), methane from bio-based material (bio-methane), biogas produced from the decomposition of organic materials or combinations thereof. Input material may be provided to at least one gasification plant, steam cracker or synthesis gas plant, or any plant of the production chain for downstream products such as nitrogen, ammonia, methanol, ethylene, propylene, sulfur or the like.

The input material associated with one or more environmental attribute(s) provided to the entry point of the chemical production network may include recycled input materials including, but not limited to, recycled pyrolysis oil, recycled pyrolysis gas, recycled synthesis gas, recycled hydrogen, recycled naphtha, recycled methane, recycled ethane, recycled propane, recycled chemicals or combinations thereof. Recycled chemicals may include, but may not be limited to, recycled ammonia, recycled methanol, recycled ethylene, recycled propylene, recycled benzene, recycled toluene, recycled xylene or combinations thereof. In the context provided here recycled input material may include any material that at least in part includes recycled content and/or is at least in part produced from recycled content. The recycled content may be physically and/or chemically traceable.

The input material associated with one or more environmental attribute(s) provided to the entry point of the chemical production network may include renewable input materials including, but not limited to, renewable pyrolysis oil, renewable pyrolysis gas, renewable synthesis gas, renewable hydrogen, renewable naphtha, renewable methane, renewable ethane, renewable propane, renewable chemicals or combinations thereof. Renewable chemicals may include, but may not be limited to, renewable ammonia, renewable methanol, renewable ethylene, renewable propylene, renewable benzene, renewable toluene, renewable xylene or combinations thereof. In the context provided here renewable input material may include any material that at least in part includes renewable content and/or is at least in part produced from renewable content. The renewable content may be physically and/or chemically traceable.

The input material associated with one or more environmental attribute(s) provided to the entry point of the chemical production network may include bio-based input materials including, but not limited to, bio-based pyrolysis oil, bio-based pyrolysis gas, bio-based synthesis gas, bio-based hydrogen, bio-based naphtha, bio-based methane, bio-based ethane, bio-based propane, bio-based chemicals or combinations thereof. Bio-based chemicals may include, but may not be limited to, bio-based ammonia, bio-based methanol, bio-based ethylene, bio-based propylene, bio-based benzene, bio-based toluene, bio-based xylene or combinations thereof. In the context provided here bio-based input material may include any material that at least in part includes bio-based content and/or is at least in part produced from bio-based content. The bio-based content may be physically and/or chemically traceable.

The chemical production network may include identity preserving or segregated production chains. Identity preserving or segregated in this context may refer to the environmental attributes of the input materials being preserved or segregated in the production chains. Examples are bio-based-renewable or recycled input materials used to produce the chemical product without fossil content. Further examples are fossil input materials used to produce the chemical products with fossil content. Chemical production networks may include non-identity preserving or non-segregated production chains. Non-identity preserving or non-segregated in this context may refer to input materials associated with environmental attributes being mixed. For example, non-identity preserving or non-segregated in this context refers to input materials associated with environmental attributes being mixed with fossil input materials in the production chains. Examples are fossil and renewable input materials mixed to produce the chemical product with fossil and renewable content.

The chemical production network may include multiple production steps per production chain. The production steps included in the chemical production network may be defined by the physical system boundary of the chemical production network. The system boundary may be defined by location or control over production processes. The system boundary may be defined by the site of the chemical production network. The system boundary may be defined by production processes controlled by one entity or multiple entities jointly. The system boundary may be defined by production or value chain with staggered production processes to an end product, which may be controlled by multiple entities separately. For example, the chemical production network may include a waste collection step, a waste sorting step, a recycling step such as chemical recycling through pyrolysis, a cracking step such as steam cracking, a separation step to separate outputs of one process step and further processing steps to convert such outputs to chemical products leaving the system boundary of the chemical production network. The entry points of the chemical production network may be marked by the entry of input materials to the chemical production network. The input materials entering the chemical production network may be used to produce one or more chemical products. The chemical products may leave the physical system boundary of the chemical production network. The exit points of the chemical production network may be marked by the exit of chemical products from the chemical production network.

The virtual balancing account may refer to a digital storage structure that stores data related to environmental attributes. The account may be associated with meta data identifying the account for balancing environmental attributes. The account may be associated with meta data identifying the environmental attributes and the environmental or balancing units allocated to the account. The account may be associated with meta data identifying the production chain the account is associated with. The account may be associated with meta data identifying the input or target material the account is associated with. The account may be part of a balancing system including multiple accounts. The account may hold environmental attributes for transaction. Environmental attributes may be allocated, added, deleted, withdrawn, or deducted from the account. The virtual balancing account may by associated with environmental attribute types such as recycled or renewable. The virtual balancing account may by associated with input material types such as pyrolysis oil, bio-naphtha, bio-methane, bio-gas or combinations thereof. The virtual balancing account associated with the environmental attribute type recycled may be further associated with waste-stream type such as mixed plastics waste, specific end product waste, e.g., tiers waste or foam waste, post-consumer waste, pre-consumer waste or combinations thereof. The virtual balancing account may by associated with an allocation scheme such as segregated allocation, non-segregated allocation like book and claim, mass balance with free attribution, mass balance without free attribution or combinations thereof.

The chemical product may be produced by the chemical production network to which the input material(s) associated with one or more environmental attribute(s) were provided. The chemical product may be produced by a production chain of the chemical production network to which the input material(s) associated with one or more environmental attribute(s) were provided. The chemical product may be produced from the input material(s) associated with one or more environmental attribute(s).

In one embodiment the environmental attribute relates to or is associated with a renewable, a bio-based and/or a recycled content. For example, input material from organic waste may be associated with the environmental attributes recycled and bio-based. Input material from organic waste may be bio-based and recycled input material. Further for example, input material from wooden waste may be associated with the environmental attributes recycled, bio-based and renewable. Input material from wooden waste may be bio-based, renewable and recycled input material. In the context provided here renewable, a bio-based and/or a recycled input material may include any material that at least in part includes renewable, a bio-based and/or a recycled content and/or is at least in part produced from renewable, a bio-based and/or a recycled content. The renewable, a bio-based and/or a recycled content may be physically and/or chemically traceable.

In another embodiment the virtual balancing system or the balancing account relates to or is associated with one or more characteristic(s) of the input material associated with one or more environmental attribute(s). The virtual balancing system or the balancing account may relate to one or more characteristic(s) of the input material that are attributable to environmental impact of the input material and the chemical product produced by the chemical production network. The characteristic(s) of the input material may include, and may not be limited to, an input material type, a waste stream type, a biomass type, a renewable type, an allocation scheme, or combinations thereof

By specifying the characteristics of the input material, the environmental attribute and characteristics of input materials used to produce the chemical product may be tracked with more granularity. This enables to track the environmental attributes and characteristics of the input material more granularly. More granular tracking via the virtual accounting system in return enables assignment of environmental attributes from balancing accounts that are tailored to customer needs.

In another embodiment the virtual balancing system or the balancing account relates to or is associated with an input material type, a waste stream type, a biomass type, a renewable type, an allocation scheme, or combinations thereof.

The virtual balancing system or the balancing account may be associated with metadata specifying the input material associated with one or more environmental attribute(s). The metadata may specify the input material associated with one or more environmental attribute(s) provided to the entry point of the chemical production network. The input material type may include, and may not be limited to, pyrolysis oil, pyrolysis gas, synthesis gas, hydrogen, naphtha, methane, ethane, propane, chemicals, or combinations thereof. Chemicals may include, but may not be limited to, ammonia, methanol, ethylene, propylene, benzene, toluene, xylene, or combinations thereof.

The virtual balancing system or the balancing account may be associated with metadata specifying an input material type, a waste stream type, a biomass type, a renewable type, an allocation scheme, or combinations thereof. As one example, recycled pyrolysis oil produced from plastics waste may be provided to the chemical production network as input material. The metadata may specify the input material type pyrolysis oil, the environmental attribute type recycled, the waste stream type mixed plastics waste, specific end product waste, post-consumer waste or pre-consumer waste, and/or the allocation scheme non-segregated scheme such as mass balance. As another example, bio-based naphtha may be provided to the chemical production network as input material. The metadata may specify the input material type naphtha, the environmental attribute type bio-based, the biomass type palm oil and/or the allocation scheme non-segregated scheme such as mass balance. As another example, bio-based methane may be provided to the chemical production network as input material. The metadata may specify the input material type methane, the environmental attribute type bio-based, the biomass type waste from agriculture and/or the allocation scheme non-segregated scheme such as mass balance.

In another embodiment the virtual balancing system or the balancing account is associated with input material types including pyrolysis oil, pyrolysis gas, synthesis gas, hydrogen, r-chemicals, bio-naphtha or bio-methane, wherein the balancing account is associated with pyrolysis or gasification and the waste stream the pyrolysis oil, pyrolysis gas, synthesis gas, hydrogen or r-chemicals is produced from, wherein the balancing account is associated with bio-naphtha or bio-methane and the renewable stream the bio-naphtha or bio-methane is produced from.

In another embodiment the virtual balancing system or the balancing account relates to or is associated with waste stream types. The waste stream types may be linked to at least the environmental attribute recycled. The waste stream types may relate to one or more waste materials or waste categories. Waste categories may include, and are not limited to, non-synthetic waste such as animal waste, vegetable or wooden waste, or synthetic waste, such as textile waste, paper waste, plastics waste, or rubber waste. The waste stream types may relate to the origin of the waste, such as the end-of-life product, the producer of the end-of-life product, the geolocation of the end-of-life product, the habitat of the end-of-life product, the consumer of the end-of-life product, or combinations thereof and may not be limited thereto. For example, in the case of recycled as environmental attribute and pyrolysis oil as input material type the waste type may specify plastics waste from tires. mixed plastics waste from packaging or mixed plastics waste from ocean cleanup.

In another embodiment the virtual balancing system or the balancing account relates to or is associated with biomass types or renewable types. The biomass type may be linked to at least the environmental attribute bio-based or renewable. The biomass type may relate to one or more raw materials the input material is produced from. Raw materials may include, and are not limited to, agricultural waste, manure, municipal waste, plant material, sewage, green waste, food waste, natural occurring fats, natural occurring oils, mixtures of natural occurring fats and oils, cooking oil, or animal fats. The biomass type may relate to the origin of the biomass, such as the producer of the biomass, the geolocation of the biomass, the habitat of the biomass, the consumer of the biomass, or combinations thereof and may not be limited thereto. For example, in the case of bio-based as environmental attribute and bio-naphtha as input material type the biomass type may specify oils and fats from plants (vegan). Further for example, in the case of bio-based as environmental attribute and biogas as input material type the biomass type may specify municipal waste.

In another embodiment the virtual balancing system or the balancing account is associated with metadata specifying the relationship between the input material(s) associated with one or more environmental attribute(s) and the chemical product. In another embodiment the relationship relates to the chemical production network, one or more production chain(s), one or more chemical product(s), one or more chemical product classes or combinations thereof.

The relationship may relate to the chemical production network. For example, the input material(s) associated with one or more environmental attribute(s) may be provided to the chemical production network producing the chemical product. The relationship may relate to one or more production chain(s) of the chemical production network producing the chemical product. For example, the input material(s) associated with one or more environmental attribute(s) may be provided to one or more production chain(s) of the chemical production network producing the chemical product. The relationship may relate to one or more chemical product class(es) produced by the chemical production network. For example, the input material(s) associated with one or more environmental attribute(s) may be provided to one or more production chain(s) of the chemical production network producing the chemical product class(es). The relationship may relate to one or more chemical product(s) produced by the chemical production network. For example, the input material(s) associated with one or more environmental attribute(s) may be provided to one or more production chain(s) of the chemical production network producing the one or more chemical product. The relationship may relate to the chemical production network, one or more production chain(s), one or more chemical product(s), one or more chemical product classes or combinations thereof. By tagging with the relationship metadata the input materials may be physically and/or chemically traceable. The metadata may be derived from a digital twin of the chemical production network. The metadata may be derived from a digital twin of the chemical production network and a production schedule.

In another embodiment at least one target environmental attribute for the chemical product is provided and based on the at least one target environmental attribute at least one balancing account for deallocation the respective environmental attribute is selected. The steps may be performed at any stage of the method. For example, assigning the at least one environmental attribute to the at least one chemical product may include providing at least one target environmental attribute for the chemical product and based on the at least one target environmental attribute selecting at least one balancing account for deallocation the respective environmental attribute. For example, at least one target environmental attribute for the chemical product may be provided and assigning the at least one environmental attribute to the at least one chemical product may include based on the at least one target environmental attribute selecting at least one balancing account for deallocation the respective environmental attribute.

Owing to the virtual accounting system and associated metadata granular target environmental attributes can be achieved. The target environmental attribute may relate to or be associated with a renewable, a bio-based and/or a recycled content. The target environmental attribute may further be related to or associated with metadata specifying an input material type, a waste stream type, a biomass type, a renewable type, an allocation scheme, or combinations thereof. Based on such metadata the metadata associated with or related to the virtual accounting system or the balancing accounts may be matched, and matching balancing accounts may be selected. The respective environmental attributes may be deallocated from the selected balancing accounts. The deallocated environmental attributes may be digitally assigned to the chemical product, e.g., by assigning the environmental attribute from the balancing account to the chemical product identifier.

In another embodiment assigning the at least one environmental attribute to the at least one chemical product includes providing at least one target environmental attribute for the chemical product,
checking the environmental attribute balance of the balancing account associated with the at least one target environmental attribute and assigning the at least one environmental attribute from the respective balancing account to the at least one chemical product, if the balance is sufficient, and/or
checking the respective balancing accounts are associated with input material(s) used to produce the chemical product and assigning the at least one environmental attribute from the respective balancing account to the at least one chemical product if the respective balancing account is associated with input material(s) used in the production chain of the chemical product. Input material(s) used to produce the chemical product may include input material(s) associated with environmental attributes at the entry to the chemical production network. The respective environmental attributes may be decoupled from the material flow by allocation to the balancing account. Input material(s) used to produce the target material may include input material(s) associated with environmental attribute(s) and input material(s) not associated with environmental attribute(s). The environmental attribute(s) may be decoupled from the material flow through the chemical production network by allocation to the balancing account.

Input material(s) used to produce the chemical product may include input material(s) provided at entry points to the chemical production network. The entry points to the chemical production network may signify any point of input material(s) entering the physical boundary of the chemical production network. Upon entry material data related to the one or more input material(s) and respective environmental attributes may be provided to a computing interface configured to allocate the environmental attribute(s) to the balancing account. The material data may include at least one material identifier and at least one environmental attribute. The material data may further include an amount of input material provided. The at least one environmental attribute may be allocated to the balancing account. This way the material flow of the input material(s) through the chemical production network may be decoupled from the environmental attributes and the environmental attributes may be allocated to chemical products fully or at least partially independent of material flows.

In another embodiment assigning at least one environmental attribute to the at least one chemical product includes providing a chemical product identifier associated with the produced chemical product and assigning the environmental attribute from the balancing account to the chemical product identifier. Assigning at least one environmental attribute associated with input material(s) to chemical product(s) may include the linking of the chemical product identifier with the environmental attribute. The chemical product identifier may be associated with the physical entity of the chemical product. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

### BRIEF DESCRIPTION OF THE DRAWINGS

FIG. 1 illustrates an example of a chemical production network producing one or more chemical product(s) from one or more input material(s) in connection with an operating system including an attribute management system.

FIGS. 2a-c illustrate a part of a chemical production network producing multiple chemical product(s) from fossil and non-fossil input material(s).

FIGS. 3a-c illustrate examples of allocation schemes allocating the use of renewable or bio-based input materials to chemical products of the chemical production network.

FIG. 4 illustrates an example of a chemical production network with different allocation schemes.

FIGS. 5a,b illustrate inbound and outbound allocation of environmental attributes for a chemical production network.

FIG. 6 illustrates an example of a method for assigning at least one environmental attribute to at least one chemical product produced by a chemical production network.

FIG. 7 illustrates an example of an apparatus for producing at least one chemical product associated with one or more environmental attribute(s) including an example method for converting and allocating to the virtual balancing account on inbound.

FIG. 8 illustrates an example of an apparatus for producing at least one chemical product associated with one or more environmental attribute(s) including an example method for converting and assigning to the chemical product on outbound.

FIG. 9 illustrates an example of chemical products produced by the chemical production network.

FIG. 10 illustrates an example of the converting to balancing units and assignment to chemical products.

FIGS. 11a-c illustrate examples of virtual balancing accounts and associated metadata.

FIG. 12 illustrates an example of a method for producing at least one chemical product associated with at least one environmental attribute for a supply chain.

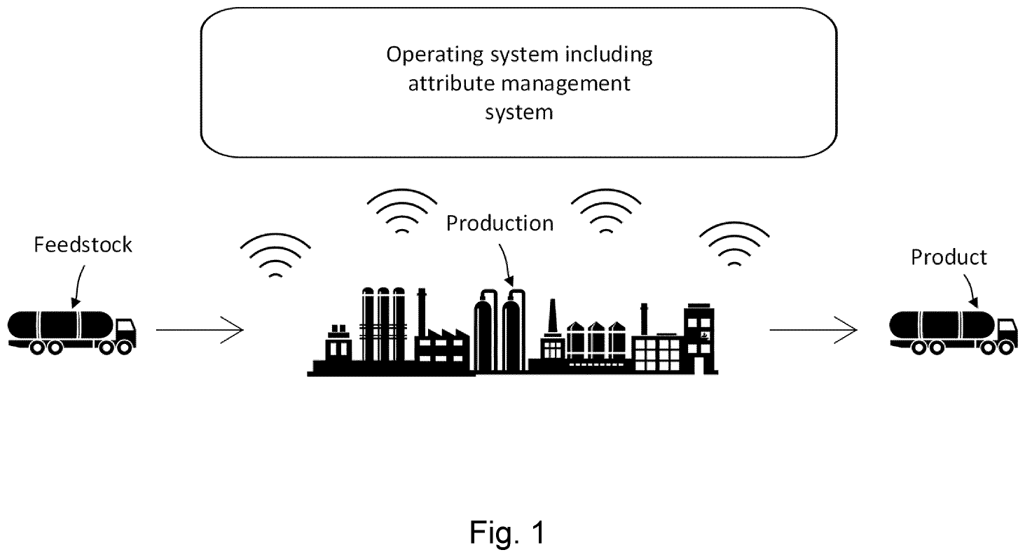

FIG. 1 illustrates an example of a chemical production network producing one or more chemical product(s) from one or more input material(s) in connection with an operating system including an attribute management system.

For producing one or more chemical product(s) different input materials (feedstocks) may be provided as physical inputs from material providers or suppliers. The chemical products produced from the input materials may have one or more properties related to the environmental impact of the input materials or the chemical products produced from the input materials, that may be signified by the environmental attributes . . . .

The chemical production network may include multiple interlinked processing steps. The chemical production network may be an integrated chemical production network with connected or interconnected production chains. The chemical production network may include multiple different production chains that have at least one intermediate product in common. The chemical production network may include multiple stages of the chemical value chain. The chemical production network may include the producing, refining, processing and/or purification of gas or crude oil. The chemical production network may include a stream cracker, or a syngas plant connected to multiple production chains that output chemical products from the effluent of the steam cracker or syngas plants. The chemical production network may include multiple production chains that produce from one or more input material(s) chemical products that exit the chemical production network. The chemical production network may include multiple tiers of a chemical value chain. The chemical production network may include physically connected or interconnected supply chains and/or production sites. The production sites may be at the same location or at different locations. In the latter case, the production sites may be connected or interconnected by means of dedicated transportation systems such as pipelines, supply chain vehicles, like trucks, ships or other cargo transportation means.

The chemical production network may chemically convert input materials via chemical intermediates to one or more chemical product(s) that exit the chemical production network. The chemical production network may convert input material(s) by way of chemical conversion to one or more chemical product(s).

The input material(s) may be fed into the chemical production network at any entry point. The input material(s) may be fed into the chemical production network at the start of the chemical production network. Input materials may for example make up the feedstock of a steam cracker. The input material may include a bio-based, a recycled and/or a fossil input material for the manufacture of chemical intermediates and chemical products.

The chemical production network may include multiple production steps. The production steps included in the chemical network may be defined by the system boundary of the chemical production network. The system boundary may be defined by location or control over production processes. The system boundary may be defined by the site of the chemical production network. The system boundary may be defined by production processes controlled by one entity or multiple entities jointly. The system boundary may be defined by value chain with staggered production processes to an end product, which may be controlled by multiple entities separately. The chemical production network may include a waste collection and sorting step, a recycling step such as pyrolysis, a cracking step such as steam cracking, a separation step to separate outputs of one process step and further processing steps to convert such outputs to a chemical product leaving the system boundary of the chemical production network.

The operating system of the chemical production network may monitor and/or control the chemical production network based on operating parameters of the different processes. One process step monitored and/or controlled may be the feed of input materials or the discharge of chemical products. Another process step monitored and/or controlled may the allocation of environmental attributes to chemical products produced via the chemical production network. Yet another process step monitored and/or controlled may be the registration of environmental attributes associated with input material(s) entering the system boundary of the chemical production network. Yet another process step monitored and/or controlled may be the management of environmental attributes associated with input material(s) and chemical product(s) of the chemical production network.

The operating system may be configured to access data related the inputs material(s), the process(es) and/or the chemical product(s) produced by the chemical production network. The operating system may be configured to convert a recycled, renewable, or bio-based content of the one or more input material(s) used in the chemical production network to balancing units. The operating system may be configured to allocate the balancing units to at least one balancing account associated with the recycled or bio-based content of the input materials. The operating system may be configured to allocate at least a part of the balancing units from the at least one balancing account to the at least one chemical product.

The operating system may be configured to handle balancing units related to the input and chemical products produced by the chemical production network. In particular, the operating system may be configured to determine balancing units associated with the use of input materials impacting the environmental property/attribute of the chemical products produced by the chemical production network. Further in particular, the operating system may be configured to determine balancing units associated with the chemical product(s) and the environmental property of the chemical product(s). This way the operating system may be configured to allocate balancing units to balancing accounts or to deallocate balancing units from the balancing accounts. The balancing units may be viewed as a credit that may be deposited in an account or deducted from an account related to the input and chemical products of the chemical production network.

The operating system may be configured to register inbound environmental attributes, to assign outbound environmental attributes and to manage inbound allocation as well as outbound assignment.

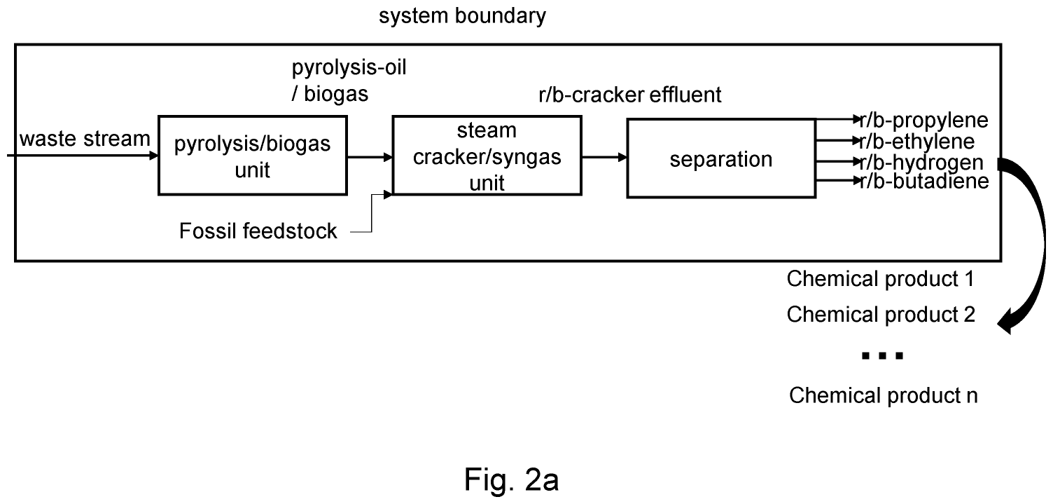

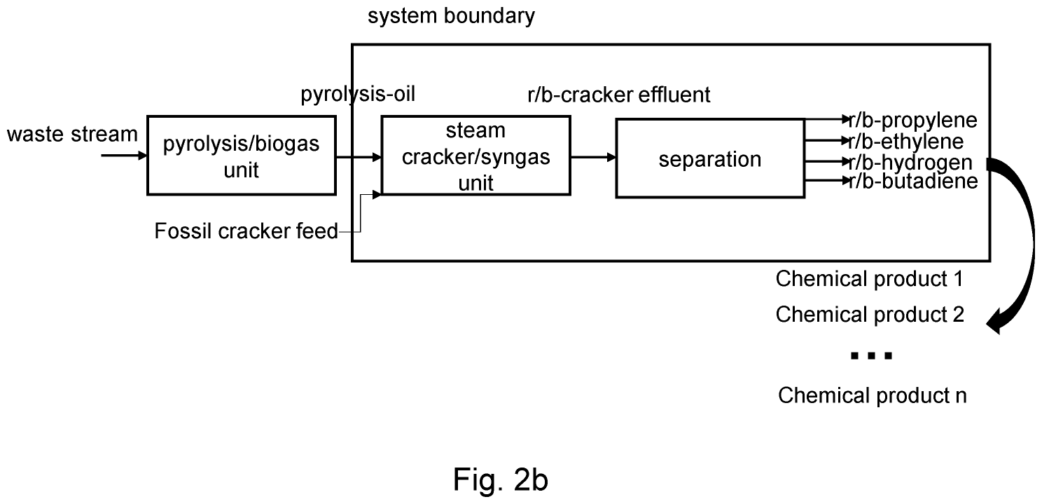

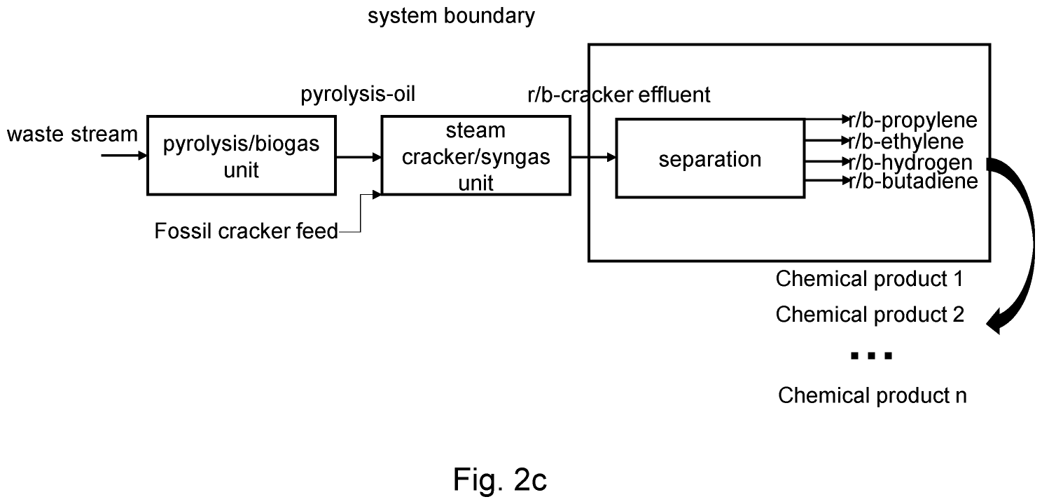

FIGS. 2a-c illustrate a part of a chemical production network producing multiple chemical product(s) from fossil and non-fossil input material(s).

The chemical production network may comprise a pyrolysis unit for pyrolysis of recycled waste. A waste stream may be fed to the pyrolysis unit. Waste stream may include plastics, rubber (including tires), textiles, wood, biowaste, modified celluloses, wet laid products, and any other material suitable for pyrolysis. The recycled waste stream may include a stream containing at least in part post-industrial, or post-consumer, or both post-industrial and post-consumer materials. A post-consumer material may be a material that has been used at least once for its intended application for any duration of time regardless of wear, or has been sold to an end use customer, or which is discarded by any person or entity other than a manufacturer or business engaged in the manufacture or sale of the material. A post-industrial material may be a material that has been created and has not been used for its intended application or has not been sold to the end use customer or discarded by a manufacturer or any other entity engaged in the sale of the material.

Examples of post-industrial materials include rework, regrind, scrap, trim, out of specification materials, and finished materials transferred from a manufacturer to any downstream customer (e.g., manufacturer to wholesaler to distributor) but not yet used or sold to the end use customer. The waste stream may be isolated as one type of waste stream with specific waste material, or it may be a stream of mixed wastes.

Examples of plastics as a waste stream include high density polyethylene and copolymers thereof, low density polyethylene and copolymers thereof, polypropylene and copolymers thereof, other polyolefins, polystyrene, polyvinyl chloride (PVC), polyvinylidene chloride (PVDC), polyesters including polyethylene terephthalate, co-polyesters and terephthalate co-polyesters (e.g. containing residues of 2,2,4,4-tetramethyl-1,3-cyclobutanediol, CHDM cyclohexanedimethanol, neopentyl glycol monomers, propylene glycol,), polyethylene terephthalate, polyamides, poly(methyl methacrylate), polytetrafluoroethylene, acrylonitrile butadiene styrene (ABS), polyurethanes, cellulosics and derivates thereof such as cellulose acetate, cellulose diacetate, cellulose triacetate, cellulose propionate, cellulose butyrate; regenerated cellulosics such as viscose and rayons, epoxy, polyamides, phenolic resins, polyacetal, polycarbonates, polyphenylene-based alloys, polypropylene and copolymers thereof, polystyrene, styrenic compounds, vinyl based compounds, styrene acrylonitrile, thermoplastic elastomers, urea based polymers and/or melamine containing polymers.

The pyrolysis effluent exiting the pyrolysis unit may comprise pyrolysis oil. The pyrolysis oil may be used as feedstock for a cracker unit or a syngas plant. The pyrolysis oil may be fed to the cracker unit as recycled feedstock together with a non-recycle or conventional feedstock (e.g., propane, ethane, naphtha and/or natural gasoline). In addition, or alternatively to the recycled feedstock, bio-based feedstock may be fed to the cracker unit. For example, bio-naphtha may be produced through hydrogenation of bio-based material such palm oil and tallow and provided as feedstock to the cracker unit. In further embodiments pyrolysis wax, pyrolysis gas, pyrolysis char, or synthesis gas may be provided to the cracker unit as feedstock.

The cracker unit may include a steam cracker that breaks saturated hydrocarbons down into smaller, often unsaturated, hydrocarbons. Steam crackers are facilities in which fossil feedstock such as naphtha, liquefied petroleum gas (LPG), ethane, propane, or butane and/or non fossil feedstock is thermally cracked through the use of steam in steam cracking furnaces or electric furnaces to produce lighter hydrocarbons.

The output effluent of the cracker unit may be a recycled and/or bio-based content effluent containing light olefins, C4 products and heavy cracker products such C5, C6, C7, C8, C9, C10 products or mixtures. The recycled and/or bio-based content cracked effluent may be subjected to separation in a separation train. The recycled or bio-based content effluent can be separated in different fractions containing recycled or bio-based content from the recycled or bio-based content of the cracked effluent.

The light olefins fraction may include ethylene and propylene. Ethylene may be used to produce polyethylene, ethylene chloride and ethylene oxide. Polyethylene, ethylene chloride or ethylene oxide may be chemical intermediates used to produce chemical product(s) that exit the chemical production network. Polyethylene, ethylene chloride or ethylene oxide may be chemical product(s) that exit the chemical production network. Polyethylene, ethylene chloride or ethylene oxide may be used for producing packaging as end product, for plastic processing or for producing end products in construction and textile production. Propylene may be used to produce polypropylene, propylene oxide, acrylic acid, or other chemical derivatives. Propylene and its derivatives may be chemical intermediates used to produce chemical product(s) that exit the chemical production network. Propylene and its derivatives may be chemical product(s) that exit the chemical production network. Propylene and its derivatives may be used for producing packaging as end product, for producing furniture as end products or for producing end products in automotive production.

The C4 fraction may contain a gas mixture comprising C4 olefins from which butadiene and isobutene may be extracted. The residue, a mixture of butene and butanes, may be used as chemical intermediate for further production processes of the chemical production network. Butadiene and its derivatives may be chemical intermediates used to produce chemical product(s) that exit the chemical production network. Butadiene and its derivatives may be chemical product(s) that exit the chemical production network. Butadiene and its derivatives may be used for producing end products such as tires, papers, plastics, rubber, petroleum, lube, or perfumes. Isobutene (Isobutylene) and its derivatives may be chemical intermediates used to produce chemical product(s) that exit the chemical production network. Isobutene (Isobutylene) and its derivatives may be chemical product(s) that exit the chemical production network. Isobutene (Isobutylene) and its derivatives may be used for producing butyl rubber and for polyisobutylene for end products such as tires, papers, plastics, rubber, petroleum, lube, or perfumes.

The heavy cracker fraction may contain 5-12 hydrocarbon atoms (e.g., C5 non-aromatics, C7/C8 mixtures, C9). C5 non aromatics may be chemical intermediates used to produce further chemical intermediate(s) such as cyclopentane- and n/i pentane mixtures or chemical product(s) that exit the chemical production network. C9 fraction, not hydrogenated or hydrogenated, C7/8 mixtures or xylol-mixtures may be chemical intermediates used to produce further chemical intermediate(s) such as hydrocarbon resins, used as a blending component for premium gasoline or used for the production of benzene.

Residues like pyrolysis oil from ethylene production may be used as chemical intermediate for the production of carbon black, as an auxiliary material in the chemical industry, as a raw material for distillation of naphthalene and indene.

Based on the recycled and/or bio-based content of the cracked effluent, the fractions from the cracked effluent and any chemical products produced from any component of such effluent may contain recycled and/or bio-based content. For example, the light olefin fraction may contain recycled and/or bio-based content. Further for example, the ethylene or propylene fraction may contain recycled and/or bio-based content. Further for example chemical products produced from the ethylene or propylene fraction may contain recycled and/or bio-based content. This way the recycled and/or bio-based content may be contained in the produced chemical intermediates, chemical product(s), or end products. As shown in FIG. 2 “r-ethylene” (recycle content ethylene) may be a composition comprising: (a) ethylene obtained from cracking of a cracker feed containing pyrolysis oil, or (b) ethylene having a recycled content value attributed to at least a portion of the ethylene. Similarly, the “r-propylene” (recycle content propylene) can be a composition comprising (a) propylene obtained from cracking of a cracker feed containing pyrolysis oil, or (b) propylene having a recycled content value attributed to at least a portion of the propylene. Both, r-ethylene and r-propylene can be summarized under the term r-olefin (recycle content olefin).

FIG. 2a illustrates an embodiment for a system boundary of the chemical production network, which includes the pyrolysis step. The waste stream forms the entry point into the chemical production network. The chemical end products form the exit point out of the chemical production network. FIG. 2b illustrates another embodiment for the system boundary of the chemical production network, which excludes the pyrolysis step. The pyrolysis oil and the fossil feed form the entry point into the chemical production network. The chemical end products form the exit point out of the chemical production network. FIG. 2c illustrates another embodiment for the system boundary of the chemical production network, which excludes the pyrolysis step and the cracking step. The cracker effluent produced at least in part from pyrolysis oil forms the entry point into the chemical production network. The chemical end products form the exit point out of the chemical production network. The chemical production networks and the system boundaries illustrated in FIGS. 2a to 2c are examples and should not be considered limiting.

In the examples shown in FIGS. 2a to 2c, the recycled and/or bio-based content may be the environmental attribute of the respective input material. In FIG. 2a, the waste stream is the input material to the chemical production network including the pyrolysis unit. The environmental attribute associated with such waste stream may include the environmental attribute type such as recycled and the waste type such as mixed plastics waste. The environmental attribute may be separated from the material flow through the chemical production network on entry of the waste stream. The environmental attribute may be allocated to the virtual balancing account associated with the respective environmental attribute. In FIG. 2b, the pyrolysis oil is the input material to the chemical production network excluding the pyrolysis unit. The environmental attribute associated with such pyrolysis oil may include the environmental attribute type such as recycled, the material type such as pyrolysis oil and the waste type such as mixed plastics waste. The environmental attribute may be separated from the material flow through the chemical production network on entry of the pyrolysis oil to the chemical production network. The environmental attribute may be allocated to the virtual balancing account associated with the respective environmental attribute. In FIG. 2b, the cracked effluent produced from pyrolysis oil is the input material to the chemical production network excluding the pyrolysis unit and the cracker unit. The environmental attribute associated with such effluent may include the environmental attribute type such as recycled, the material type such as effluent produced from pyrolysis oil and the waste type such as mixed plastics waste. The environmental attribute may be separated from the material flow through the chemical production network on entry of the cracked effluent to the chemical production network. The environmental attribute may be allocated to the virtual balancing account associated with the respective environmental attribute. By separating the environmental attribute from the material flow of the chemical production network, a reliable and simple assignment of environmental attributes to the chemical products produced by the chemical production network can be realized. By way of virtual balancing accounts, the environmental attributes can be collected on entry to the chemical production network. On exit of chemical products, the environmental attributes collected on entry can be assigned to such products and de-allocated from the virtual balancing account. Thus, it can be ensured 1) that only environmental attributes are assigned to chemical products that entered the chemical production network and 2) that are not already assigned to another chemical product. As a result, the positive environmental impact is only counted once and can be tracked from entry to exit of the chemical production network.

In the context of this example a pyrolysis unit followed by a steam cracker unit is shown. This is not to be considered limiting. The concepts disclosed herein equally apply to equivalent chemical value/production chains such syngas plant-based value/production chains.

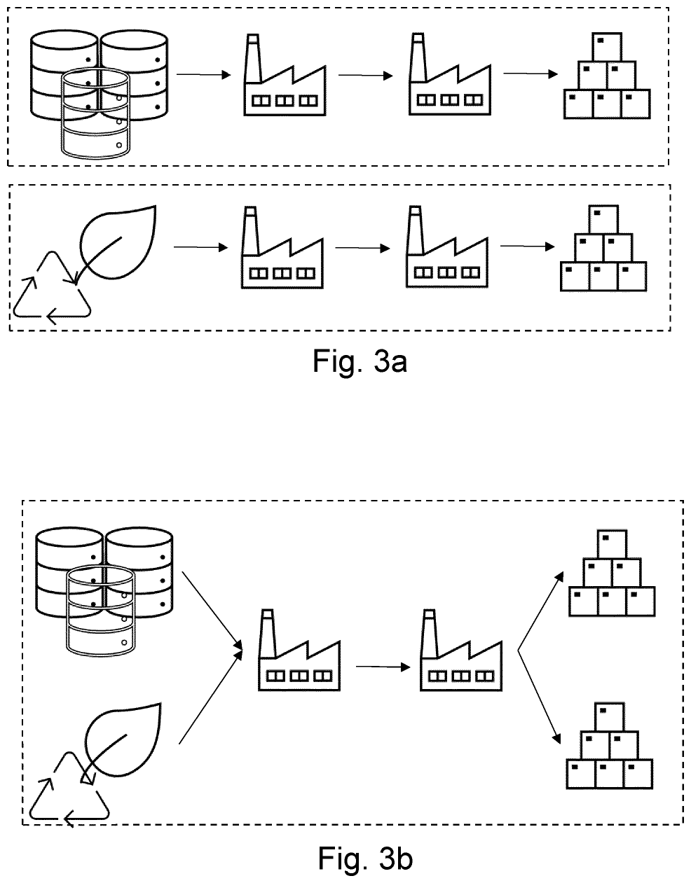

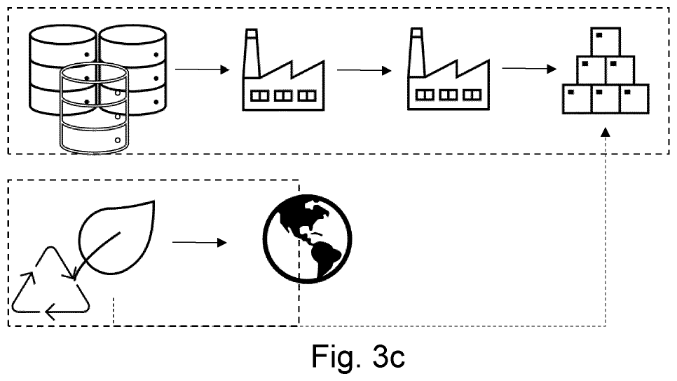

FIGS. 3a to c illustrate examples of allocation schemes allocating the use of renewable or bio-based input materials to chemical products of the chemical production network.

As illustrated in FIGS. 1 and 2 chemical production networks can comprise complex interconnected production sites that chemically convert one or more input materials via chemical processing to one or more chemical products. To account for the use of recycled or renewable content in chemical production, allocation rules may be used. This way recycled, renewable or bio-based content of input materials may be allocated to chemical products. The renewable content may be based on input material from renewable sources. The renewable content may comprise bio-based input materials produced from living organisms such as different types of crops, wood, or algae. The recycled content may comprise any recycled material used in production of new materials. This may include any recycled bio-based or bio-based materials e.g., as produced from chemical or mechanical recycling.

Accounting principles for allocating the use of recycled or renewable content are for example defined in ISO 22095. Four different chain of custody models may be used: Identity preservation models, segregated models, mass balance models, or book and claim models.

FIG. 3a illustrates an example of a dedicated or segregated production network. The production network comprises a first production chain for producing the chemical product(s) from fossil material(s) and a second production chain for producing the chemical product from bio-based input material(s). The first and the second production chain are not interconnected. The first and the second production chain produce fossil-based and bio-based or recycled chemical product(s), respectively. Example for such dedicated production environments include fermentation or chemical transformation, such as polyethylene production from sugar cane, bio-poly lactic acid (PLA) production from corn, bio-succinic acid, or bio-butanediol (BDO).

FIG. 3b illustrates an example of a complex production network. In contrast to the production network of FIG. 3a the fossil-based input material(s) are co-fed and mixed with bio-based or recycled input materials. The production network produces via one or more chemical process chain(s) with intermediates one or more material outputs or products. For the sake of simplicity FIG. 3b illustrates the mass balancing approach for one chemical process chain producing one chemical product or product. In the mass balancing model, the physical mixing or co-feeding of bio-based or recycled input material with conventional fossil input materials is accounted for. Here the feed into the production network and the feed of output products form a system boundary. The mass balance of input and chemical products connects the used bio-based or recycled input material to the produced output product. Mass balance allows to keep track of the total amount of input material (e. g. recycled or bio-based or bio-based materials) throughout the production network and allows for allocation to chemical products. Materials with different sets of specified characteristics may be mixed. E.g. recycled or bio-based feedstock replaces an equivalent amount of fossil feedstock at the beginning of the value chain (input material) and is allocated to a product (chemical product) in such a manner that the input and output match. For this model, the proportion of the input with specified characteristics might only match the initial proportions on average and will typically vary across different outputs. This means that e.g., recycled and fossil input materials are mixed and that the chemical or technical proportions in each chemical product are not tracked.

Mass balance may include conversion factors to ensure the amount of input material is correlated with the amount of chemical product. The calculation may be made over a pre-defined or specified time period. Mass balance may be based on a balancing unit such as mass, energy, or carbon.

FIG. 3c illustrates a complex production network associated with a book and claim scheme. In book and claim scheme, the characteristic renewable or recycled input material is not linked to the actual material flows. Book & Claim allows to de-couple specific characteristic, such as renewable, from the physical product and to transfer the characteristic separately via a dedicated registry in the form of a digital asset. This approach may be used for renewable energy. Book and claim may be based on a book and claim accounting unit such as kilowatthours for electricity.

In identify preservation models or segregated approaches as illustrated in FIG. 3a renewable, recycled or bio-based input materials may not be mixed with fossil input material. In mass balance or book and claim approaches as illustrated in FIGS. 3b and 3c renewable or recycled or bio-based materials with fossil input material may be mixed. In view of the increasing number of different sources for more sustainable chemical production and the number of allocation schemes, an efficient and robust operation system for operating complex production networks like chemical production networks is required.

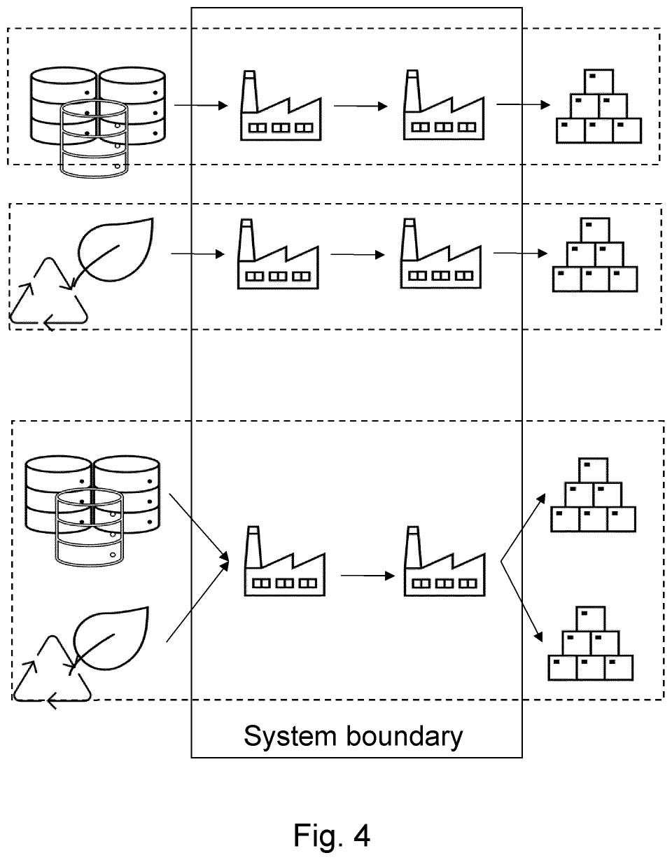

FIG. 4 illustrates an example of a chemical production network with different allocation schemes.

The chemical production network may include multiple production chains with different allocation schemes. The system boundary of the chemical production network may be defined by the entry points to the chemical production network and the exit points from the chemical production network. The production chains may be defined by the chemical product(s) produced via such production chains. The production chain logic may be based on process data associated with process steps from input material(s) to chemical product(s). For each production chain an allocation scheme may be applicable or assign to.

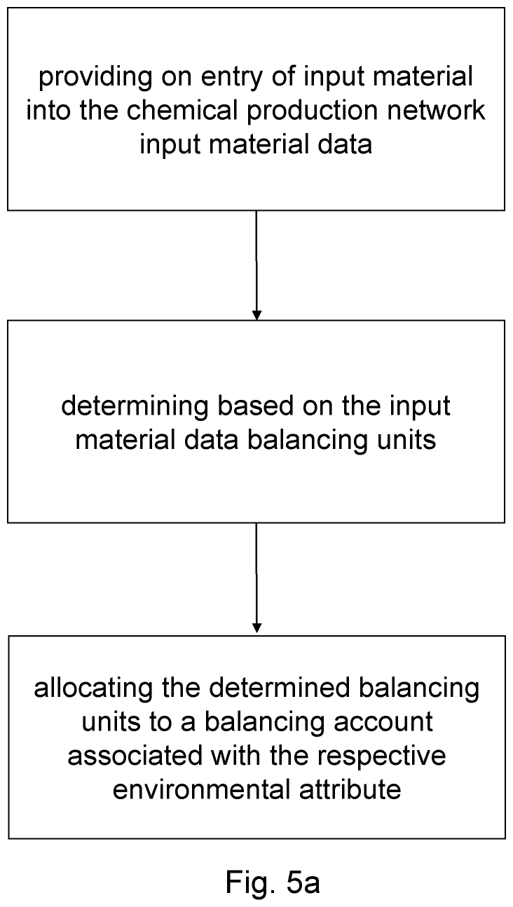

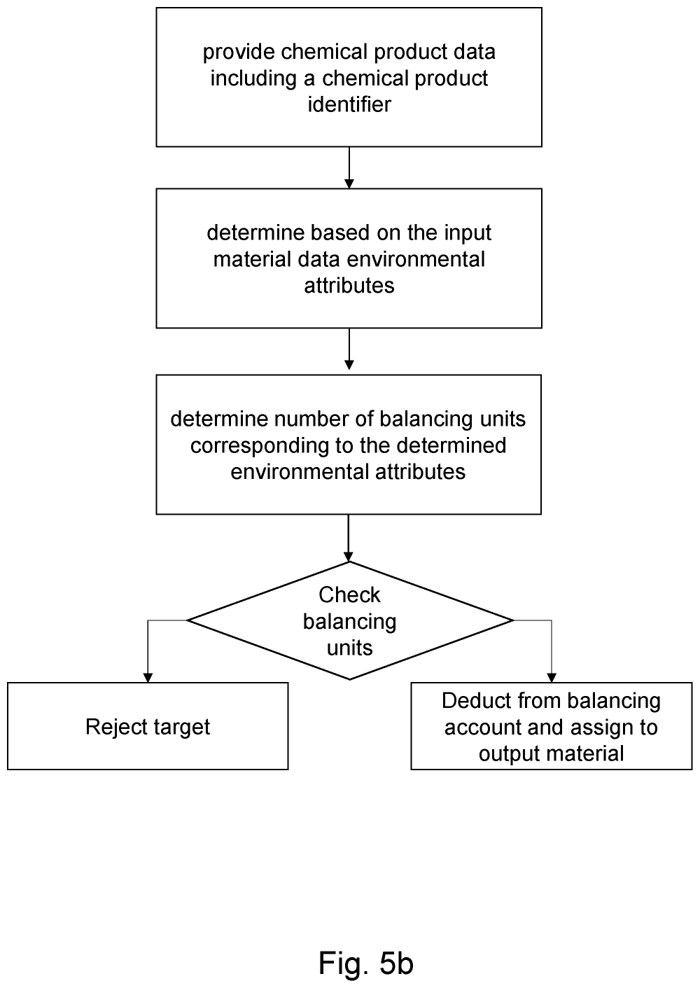

FIGS. 5a,b illustrate inbound and outbound allocation of environmental attributes for a chemical production network.

FIG. 5a illustrates an example of an inbound allocation process for the chemical production network.

As for example illustrated in FIG. 1 the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 3a-c. The chemical production network may include the network as for example described in the context of FIG. 2 or 4.

On input material(s) entering the chemical production network or the physical system boundary of the chemical production network the inbound registration or allocation takes place. In this context input materials signify any materials that enter the system boundary of the chemical production network and is used to produce chemical product(s) leaving the chemical production network. Such input material(s) may be associated with an input material identifier. Possible identifiers include decentral identifiers, input material specification(s), input material order number(s), input material LOT number(s), input material batch number(s), input material supplier specification(s) or combination thereof.

On entry of the input material(s) to the chemical production network, input material data may be provided. The input material data may include input material identifier(s), input material amount and/or one or more environmental attribute(s) associated with the input material(s).

Based on the input material data, in particular the input material amount and/or one or more environmental attribute(s) associated with the input material, balancing units may be determined by converting the input material amount to balancing units for the respective environmental attribute. The balancing units may relate to mass, weight, carbon atoms, hydrogen atoms, methane equivalents, energy property or any other suitable measure to quantify the environmental impact of the environmental attribute.

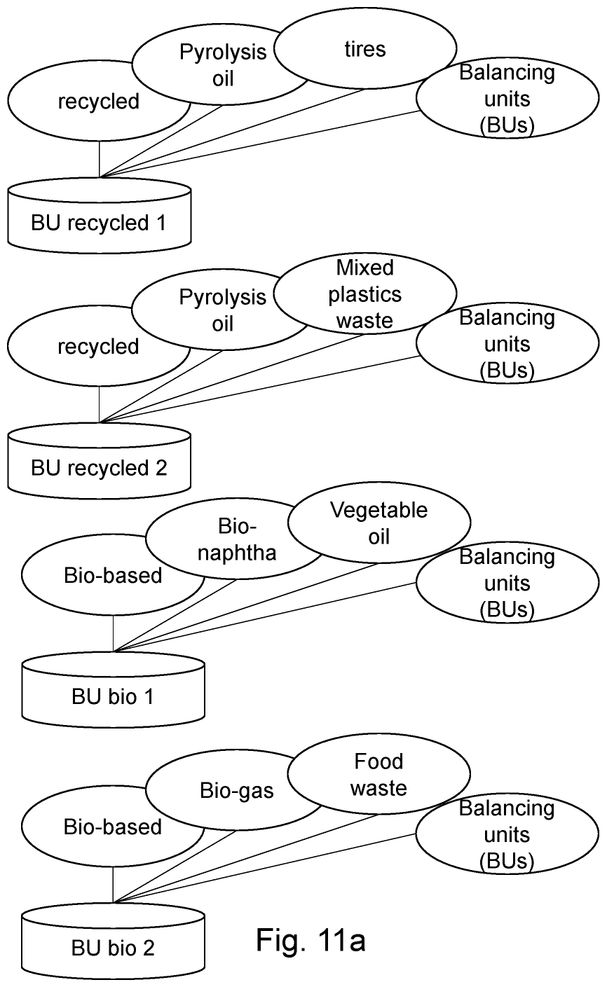

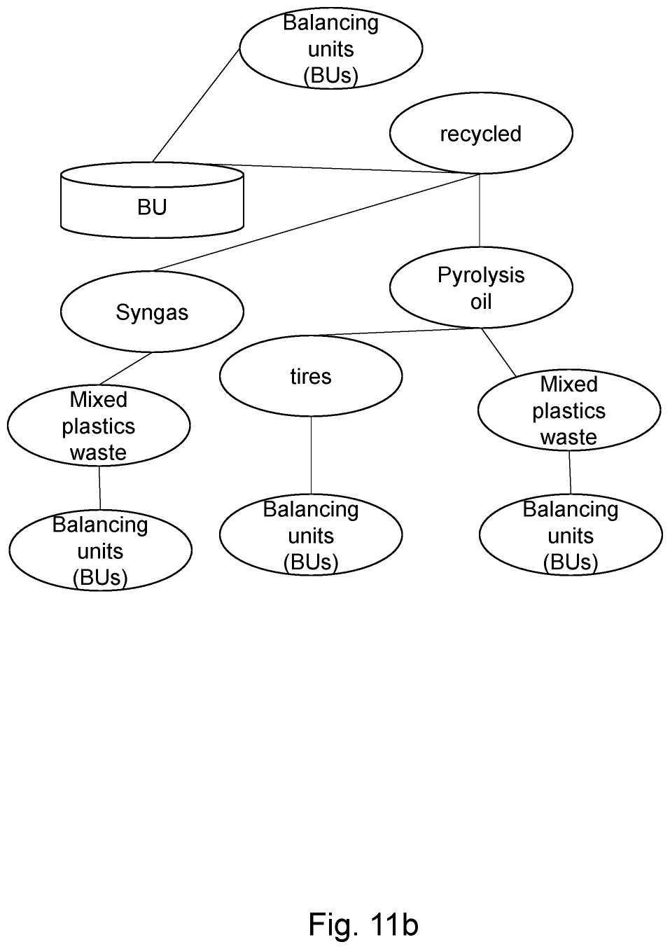

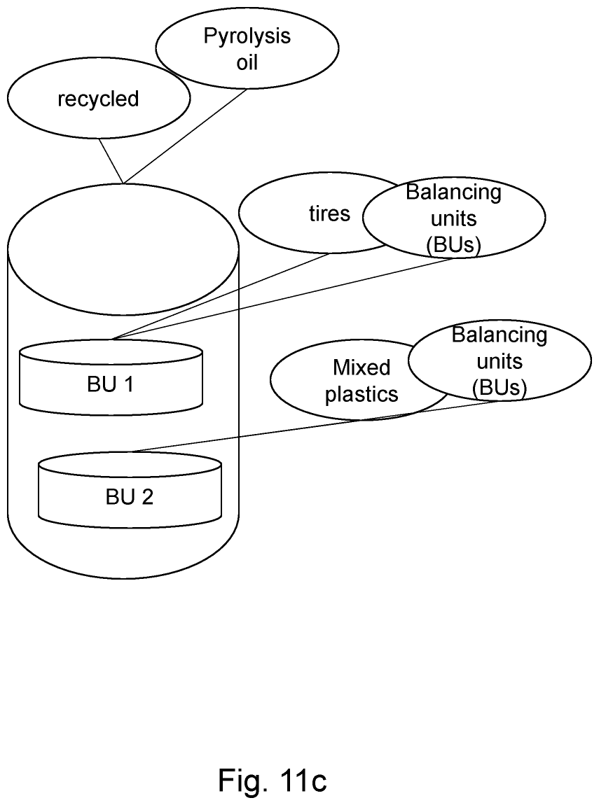

The balancing units may be allocated to a balancing account associated with the respective environmental attribute. This way the environmental attributes associated with input material(s) entering the chemical production network can be tracked or stored in an accounting system. An example of a virtual accounting system is shown in FIGS. 11a-c, where each balancing account is associated with metadata signifying the input material such as pyrolysis oil, environmental attribute type such as recycled and waste type such as tires.

For allocation the input material data may include respective metadata for the input material. Allocation rules may include instructions to allocate the environmental attribute(s) included in the input material data to the respective balancing account. In the inbound process such allocation rules may be used to allocate environmental attributes from the input material data to the virtual accounting system.

For example, the method for allocating balancing units may be associated with pyrolysis oil as input material to a complex chemical production network.

First the amount and type of waste stream may be provided to a computing interface configured to allocate the environmental attribute. The quantity and type of waste stream may be stored in connection with the pyrolysis plant(s) and pyrolysis oil produced by the plant. For instance, a pyrolysis oil producer may operate multiple plants and may track the amounts and type of waste streams fed to the plants. The waste may be provided to the pyrolysis plant separately according to type of waste or the waste may be mixed. Based on such tracking the quantity of pyrolysis oil and type of waste stream may be allocated to the produced pyrolysis oil.

The amount of pyrolysis oil and the type of waste stream may be provided to the operating system of a complex chemical production network. Based on the amount of pyrolysis oil balancing units may be generated. Such generation may include a conversion factor that takes account of the chemical difference between fossil-based input materials, such as naphtha and methane, and non-fossil input materials, such as pyrolysis oil. The conversion factor may relate to the lower heating value of the pyrolysis oil in relation the lower heating value of naphtha or methane. The conversion factor may include the ratio of the lower heating value of pyrolysis oil to naphtha or methane. This way the chemical difference between the fossil and the recycled input material can be taken into account. Pyrolysis oil may be mixed with fossile input material and the fraction of pyrolysis oil may be registered in the account for balancing environmental units.

The balancing units may be allocated to the respective balancing account(s). For example, the allocation rule may be associated with balancing account 1 of FIG. 11a. In such a case the balancing units for the pyrolysis oil produced from waste of the waste stream type tires and used in a specified amount in the production network will be allocated to balancing account 1. On production of the chemical products such balancing units may be allocated to the chemical product according to the allocation rule. The allocation rule that specifies free attribution between production processes of the chemical production network may provide for reliable allocation in complex production networks. In such case the allocation of balancing units may be decoupled from the material flows of the chemical production network.

FIG. 5b illustrates an example of an outbound allocation process for the chemical production network.

As for example illustrated in FIG. 1 the chemical production network may be operated by the operating system configured to register inbound environmental attributes, to assign outbound environmental attributes and/or to manage inbound registration as well as outbound assignment via allocation rules. The allocation rules may include allocation schemes as illustrated in FIGS. 3a-c. The chemical production network may include the network as described in the context of FIG. 2 or 4.

The chemical product may be produced from input material that entered the chemical production network. Chemical product data including the chemical product identifier may be provided. The chemical product data may further include input material data associated with the input materials used to produce the chemical product(s), process data associated with the production chain for producing the chemical products and/or data related to the chemical product data, such as a chemical product specification or a chemical product amount.

Based on the input material data environmental attributes associated with the input material(s) used to produce the chemical product(s) may be determined. The input material data may specify the total amount of input material(s) that entered the production chain for producing the amount of chemical product. The input material data may further specify the environmental attributes available for the respective input material(s). From the total amount of input material(s), the number of balancing units for respective environmental attributes available for such input material(s) may be determined. This way the maximum number of balancing units for the respective environmental attribute attributable to the chemical product may be determined.

Based on the environmental attribute(s) attributable to the chemical product, the number of balancing units corresponding to the determined environmental attributes may be determined. The determined balancing units may be compared to balancing units stored in balancing account(s) for the respective environmental attribute(s).

If the balance of the respective balancing account(s) for the respective environmental attribute(s) is not sufficient, the environmental attribute(s) is rejected. If the balancing units are available, the balancing units are deducted from the respective balancing account(s) and the environmental attribute(s) are assigned to the chemical product.

An example of an accounting system is shown in FIGS. 11a-c, where each balancing account is associated with metadata signifying the input material such as pyrolysis oil, environmental attribute type such as recycled and waste stream type such as tires. For assignment of the environmental attribute(s) to the chemical product, allocation rules may be defined. Allocation rules may include instructions to assign the environmental attribute(s) included in the input material data from the respective balancing account to the chemical product(s). For the outbound process such allocation rules may be used to assign environmental attributes from the virtual balancing system to the chemical product(s).

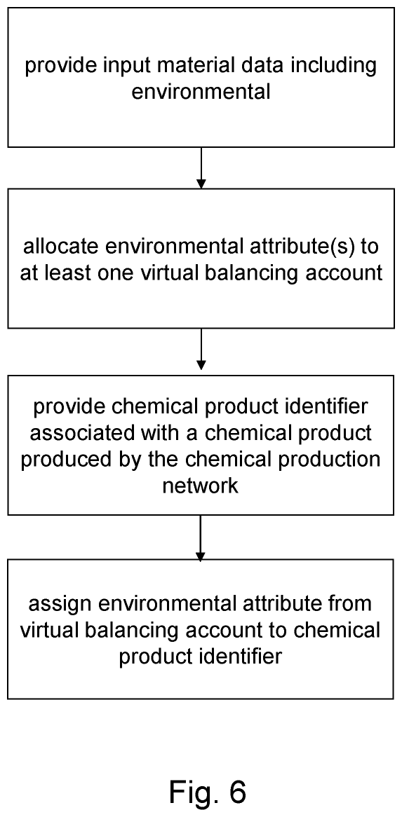

FIG. 6 illustrates an example of a method for assigning at least one environmental attribute to the least one chemical product produced by a chemical production network.

The one or more input material(s) may be provided at entry point(s) of the chemical production network. Material data related to the one or more input material(s) and respective environmental attributes may be provided to a computing interface configured for allocation and/or assignment of environmental attributes. The material data may be provided on, prior or after entry of the one or more input material(s) to the chemical production network. The material data may include the input material identifier associated with the respective input material(s) provided to the chemical production network. The input material identifier may be associated with the physical entity of the input material(s). This way the virtual identifier of the input material may be uniquely linked to the physical input material(s). Such linking may include a physical or virtual link of identifiers uniquely associated with the physical input material(s). For physical linking a tag or code may be physically connected to the material, e.g., by printing a QR code on the packaging. For virtual linking different identifiers associated with the physical material may be linked. For example, an order number, a batch number, LOT number or a combination thereof may be linked. The material data may include at least one environmental attribute associated with the respective input material and an amount of input material provided to the entry point of the chemical production network.

The one or more environmental attribute(s) may be allocated to at least one balancing account associated with the respective environmental attribute. The balancing account may be associated with a digital storage structure that stores data related to environmental attributes. The account may be associated with metadata identifying the account. The account may be associated with metadata identifying the environmental attributes and the environmental or balancing units allocated to the account. The account may be associated with metadata identifying the production chain the account is associated with. The account may be associated with metadata identifying the input or chemical product the account is associated with. The account may be part of a virtual accounting system including multiple accounts. The account may hold environmental attributes for transaction. Environmental attributes may be allocated, added, deleted, withdrawn, deallocated, or deducted from the account. The virtual balancing account as for instance illustrated in FIG. 11c may by associated with environmental attribute types such as recycled or renewable. The virtual balancing account may by associated with input material types such as pyrolysis oil, bio-naphtha, bio-methane, or the like. The virtual balancing account associated with the environmental attribute type recycled may be further associated with the waste stream type such as mixed plastics waste, specific end product waste, e.g., tires waste or foam waste, post-consumer waste, pre-consumer waste or the like. The virtual balancing account may by associated with an allocation scheme such as segregated allocation, non-segregated allocation e.g., book and claim, mass balance with free attribution, mass balance without free attribution or combinations thereof.

The chemical product identifier associated with a chemical product produced by the chemical production network and provided at the exit point from the chemical production network may be provided. The chemical product identifier may be associated with the physical entity of the chemical product. This way the virtual identifier of the material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

At least one environmental attribute from the at least one balancing account associated with the respective environmental attribute may be assigned to the at least one chemical product identifier. This way the chemical product may be provided together with the digital asset of the environmental attribute and the positive environmental impact associated with the chemical product can be tracked through the value chain using such chemical product. The tracking may stretch as far as the end product produced from the chemical product, re-use of the end product or recycling of the end product.

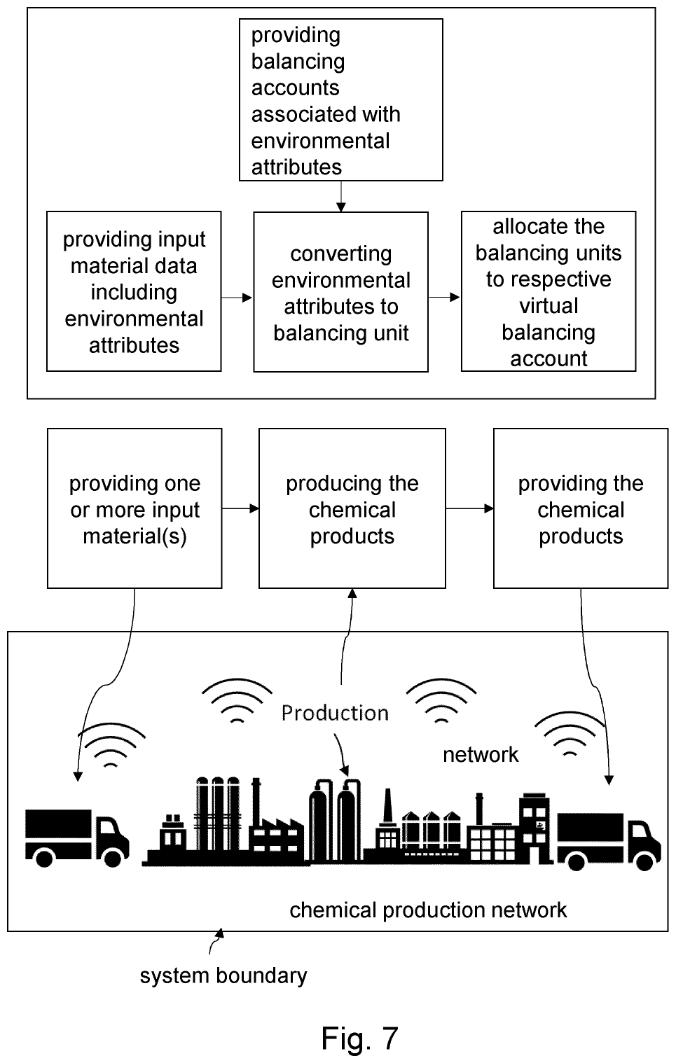

FIG. 7 illustrates an example of an apparatus for producing at least one chemical product associated with one or more environmental attribute(s) including an example method for converting and allocating to the virtual balancing account on inbound.

The input materials such as pyrolysis oil, bio-naphtha or bio-gas may be provided to the chemical production network. The input materials may enter the system boundary of the chemical production network at the entry point, such as a such as a steam cracker or a syngas plant. The input materials may be used in the chemical production network to produce one or more chemical product(s) from the input materials. Chemical product(s) may be provided on exit points of the chemical production network. Chemical products may be MDI, TDI, PA6, EPS, PC, Polyols, Caprolactam, adipic acid, HMD, Polyamides.

On entry of the input material input material data may be provided via a network to the interface of the operating system. A data provider may be configured to provide material data related to the one or more input material(s) and respective environmental attributes to a computing interface configured to allocate the environmental attributes. The material data may be provided on, prior or after providing of the one or more input material(s) at entry points to the chemical production network. The input material identifier may be associated with the physical entity of the input material entering the chemical production network.

The data related to the input material identifier may include the environmental attribute associated with the respective input material, the amount of input material and the certificate certifying the environmental attribute. The amount of input material may be a measured amount of input material fed to a plant or storage of the chemical production network for producing one or more chemical product(s) from the input material(s). The input material identifier associated with the respective input material, the environmental attribute(s) associated with the respective input material(s) and the amount of input material(s) provided to the chemical production network may be provided. Such data may be provided via a communication network on entry to the system boundary, or the data may be transferred from a computing system to the operating system.

An inbound allocator may be configured to convert and allocate the one or more environmental attribute(s) to at least one balancing account associated with the respective environmental attribute. For allocation the one or more environmental attribute(s) may be converted to balancing units and the balancing units may be allocated to the balancing account. The one or more balancing unit(s) may be allocated to the at least one balancing account associated with the respective environmental attribute. The conversion may be based on a conversion factor such as mass, weight, carbon atoms, hydrogen atoms, methane equivalents or any other suitable measure for quantifying the environmental impact of the environmental attribute. By using balancing unit(s) and conversion it can be ensured that environmental attributes of input materials are only used once for assignment to chemical products. This way double counting on input or output is avoided and the positive environmental impact can be reliable assigned to chemical products.

Recycled, renewable or bio-based content used in chemical production of one or more chemical product(s) may be converted to balancing units and allocated to balancing accounts to track such content. For conversion of recycled, renewable, or bio-based content, input material data associated with input material(s) and including environmental attribute may be provided. The input material data may relate to input material impacting the environmental property of the chemical product(s) produced by the chemical production network such as recycled or bio-based input material(s). The input material data may relate to the amount of input material impacting the environmental property of the chemical product(s) produced by the chemical production network. The amount may be provided as absolute value e.g., in kt per specified time frame such as kt/a. The quantity may be provided as relative value e.g., in % of the input material(s).

The input material data may relate to an energy value of the input material impacting at least one environmental property of the chemical production network or the energy value may be provided via a data base. The input material data may indicate an availability of input material impacting at least one environmental property of the chemical products produced by the chemical production. As an example, the input material data may include:
Input material 1 cracker: bio-naphtha
Input material quantity: x %.
Input material stock: x kt
Input material LHV: x MJ/kg
Input material 2 cracker: pyrolysis oil
Input material quantity: x %
Input material stock: x kt
Input material LHV: x MJ/kg

The one or more environmental attribute(s) may be converted to one or more balancing unit(s). In the example above, the environmental attribute associated with the input material 1 may be bio-based and the environmental attribute associated with input material 2 may be recycled. The conversion of the environmental attribute to balancing units may be based on the conversion factor relating conventional input material(s) to input material(s) associated with one or more environmental attribute(s). In the example above, the conversion factor may relate conventional naphtha based to bio naphtha or pyrolysis oil. The conversion factor may relate to the use of conventional input material(s) to the use of input material(s) associated with one or more environmental attribute(s) e.g., via mass or energy property. The conversion factor may hence take into account the difference between producing chemical products from conventional input material(s) and producing chemical products from non-conventional input material(s) or producing chemical products from a mix of conventional and non-conventional input materials. The conversion factor may relate to differences in chemical and/or physical properties of conventional and non-conventional input material(s).

In chemical production networks multiple value chains may be linked. Additionally different input materials impacting the environmental property of chemical products produced by the chemical production network may be used. Owing to the processing of chemicals in continuous or semi-continuous production and the complexity of chemical production networks, traceability of the input materials through the network may be hampered. In such scenarios an equivalent amount of input materials impacting the environmental property of chemical products produced by the chemical production network may be allocated to the virtual balancing account and assigned to one or more chemical product(s) of the chemical production network. This way certain input materials associated with at least one environmental attribute may replace an equivalent amount of fossil input materials and may be allocated to the virtual account or assigned to one or more chemical product(s). The environmental attributes may hence be decoupled from the physical material flow inside the chemical production network. Decoupling may be based on the mass balance model in that the equivalent amount assigned to the one or more chemical product(s) may not exceed the equivalent amount provided by input materials. If an equivalent amount has been allocated to the virtual account of one environmental attribute type, it may not be allocated a second time to another virtual account of the one environmental attribute type. Environmental attribute types may be recycled, bio-based, renewable or the like.

The most common examples of input materials impacting at least one environmental property of chemical products produced from such input materials are recycled, renewable or bio-based input materials. These may be used as feedstock for a cracker such as a steam cracker that breaks saturated hydrocarbons down into smaller, often unsaturated, hydrocarbons for producing the lighter alkenes such as olefins, including ethene (or ethylene) and propene (or propylene).

Recycled, renewable or bio-based input materials may have the same chemical properties as their fossil counterparts. They may be totally interchangeable and the input material quantities of e.g., naphtha or natural gas, may be substituted with the quantities of input materials associated with environmental attributes. For instance, Naphtha is commonly cited with a lower heating value (LHV) of about 44.3 MJ/kg and bio-naphtha is commonly cited with a similar LHV. In such scenario the energy based balancing unit may be about 1, meaning one unit naphtha corresponds to one unit bio-naphtha. The equivalent amount of input materials associated with the environmental attribute may be allocated in one-to-one correspondence.

However, some recycled, renewable, or bio-based input materials may have different carbon content, energetic value, or other chemical properties different to the fossil feedstocks to be replaced. This may lead to higher or lower requirements for recycled, renewable or bio-based input materials compared to the quantities of fossil feedstocks. In such situations, equivalent quantities of recycled, renewable, or bio-based input materials may not adhere to a one-to-one correspondence. For instance, a conversion factor based on the lower heating value (LHV) of fossil and bio-feedstocks as an approximation of the chemical properties may be used. Bio-gas is commonly cited with an LHV of 50 MJ/kg. In such scenario the balancing unit may be about 0.88, meaning one unit naphtha corresponds to 0.88 unit bio-naphtha. Hence the equivalent amount of input material(s) associated with at least one environmental property may be allocated according to the conversion factor of 0.88 in this scenario.

Different approaches exist to determine the conversion factor from conventional input material unit to balancing unit or equivalent assignable to one or more chemical products from chemical production. Conversion may depend on
mass such as mass of input material, mass equivalent of a molecular unit such as methane or a molecular weight,
an element, such as a number or a mole of one or more element(s) or molecular unit(s) or
an energy property such as heating value like lower or higher heating value.

By way of balancing units, the environmental attribute associated with the input material may be allocated to the virtual balancing account associated with the respective environmental attribute. For allocation, allocation rules mapping the environmental attribute to the virtual balancing account may be provided. The allocation rules may depend on the input material type and the environmental attribute type. The allocation rule may be linked to metadata relating to input material type and environmental attribute type. Similarly, the virtual balancing account may be linked to metadata relating to input material type and environmental attribute type. Based on the input material type and the environmental attribute type, the virtual balancing account the respective metadata may be matched, and the input material account may be selected for allocation.

The balancing units may be allocated to the selected virtual balancing account. For example, the allocation rule may be associated with the account for pyrolysis oil. On production of the chemical products corresponding balancing units may be assigned to the chemical products. The allocation rule may specify the assignment of balancing units to the chemical product.

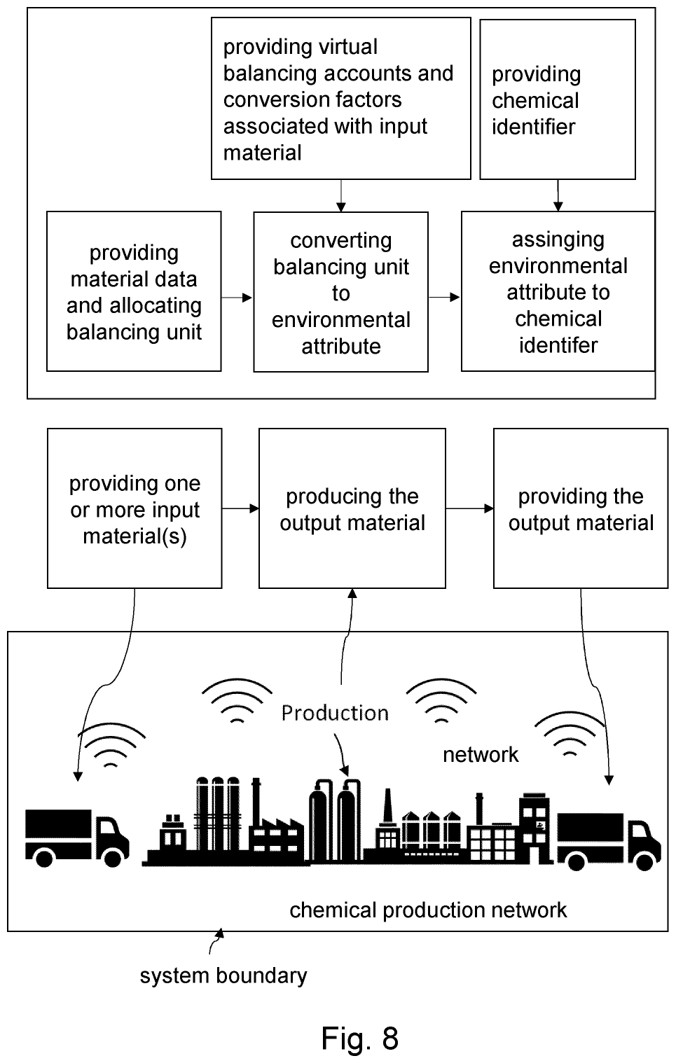

FIG. 8 illustrates an example of an apparatus for producing at least one chemical product associated with one or more environmental attribute(s) including an example method for assigning to the chemical product on outbound.

Similar to FIG. 7, the input materials such as pyrolysis oil, bio-naphtha or bio-gas may be provided to the chemical production network. The input materials may enter the system boundary of the chemical production network at the entry point, such as a such as a steam cracker or a syngas plant. The input materials may be used in the chemical production network to produce one or more chemical product(s) from the input materials. Chemical product(s) may be provided on exit points of the chemical production network. Chemical products may be MDI, TDI, PA6, EPS, PC, Polyols, Caprolactam, adipic acid, HMD, Polyamides.

Similar to FIG. 7, the one or more environmental attribute(s) may be converted and allocated to the virtual balancing account on inbound.

An identifier provider may be configured to provide the chemical product identifier associated with a chemical product produced by the chemical production network and provided at the exit point from the chemical production network. The chemical product(s) may be any chemical product produced by the chemical network.

An outbound assigner may be configured to assign at least one environmental attribute from the at least one virtual balancing account associated with the respective environmental attribute to the at least one chemical product identifier. One or more environmental attribute(s) may be assigned to the at least one chemical product. Assignment may include converting the one or more balancing unit(s) to one or more environmental attribute(s), wherein the one or more balancing unit(s) are de-allocated from the at least one virtual balancing account associated with the respective environmental attribute.

Assigning at least one environmental attribute to the at least one chemical product may include converting the one or more balancing unit(s) to one or more environmental attributes(s), wherein the one or more balancing unit(s) are deallocated from at least one virtual balancing account associated with the respective environmental attribute. Assigning at least one environmental attribute to the at least one chemical product may include providing a chemical product identifier associated with the produced chemical product and allocating the environmental attribute to the chemical product. Assigning at least one environmental attribute associated with input material(s) to chemical product(s) may include the linking of the chemical product identifier with the environmental attribute. The chemical product identifier may be associated with the physical entity of the chemical product. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

For illustration purposes and to further explain the methods shown in FIGS. 6-8, FIG. 9 shows an example of chemical products produced by the chemical production network from input materials through different intermediates.

The chemical production network may include multiple production chains with one or more intermediate stage. The production chains may have one common starting point as for example provided by the steam cracker. The cracker feedstock may comprise fossil and non-fossil input materials. The fossil input materials may comprise gaseous or liquid hydrocarbon containing feedstock such as naphtha and/or low-pressure gas or ethane. The non-fossil input materials may comprise bio-based and/or recycled feedstock.

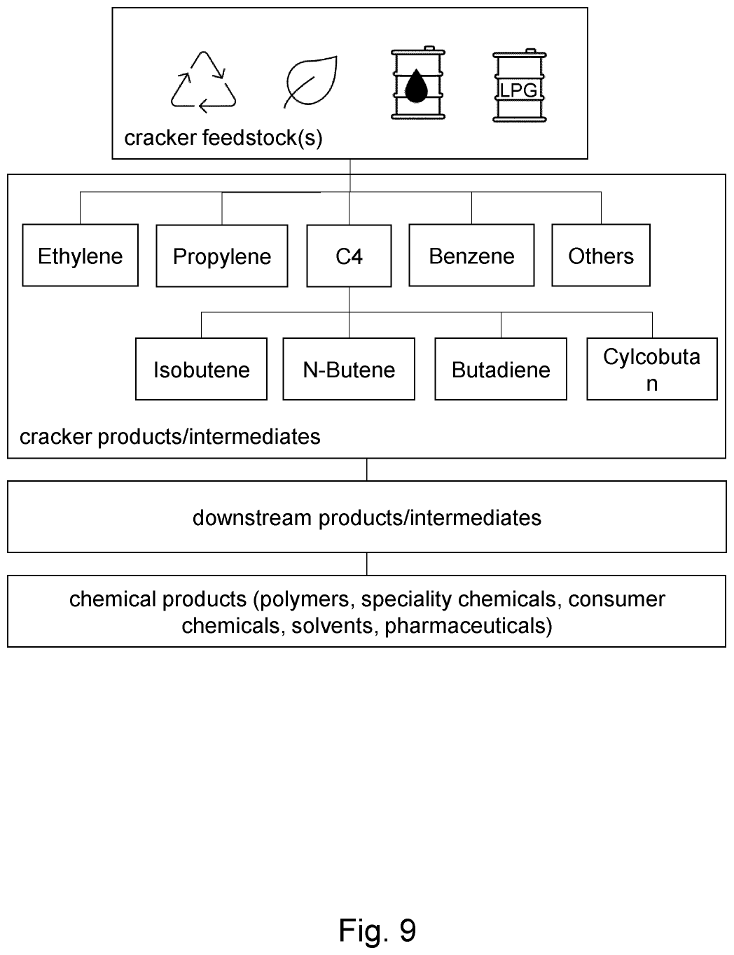

In steam cracking the feedstock is diluted with steam and heated in a furnace to reaction temperatures between 500-1000° C. After the cracking, the gas is quenched to stop the reaction in a transfer line exchanger. The cracker products produced in the reaction depend on the composition of the feed, on the hydrocarbon to steam ratio and on the cracking temperature and furnace residence time. In the example shown in FIG. 9, cracker products include light alkenes such as ethylene or propylene, C4-hydrocarbons such as Isobutene, N-Butene, Butadiene or Cyclobutane, and/or other hydrocarbons such as aromatic hydrocarbons or mixtures of aromatic hydrocarbons, such as benzene, toluene and/or xylenes (benzene and toluene also abbreviated as BT; benzene, toluene and xylenes also abbreviated as BTX).

The cracker products may be provided to the exit point of the chemical production network. The cracker products may in such embodiment be chemical products. The environmental attributes of the recycled or bio-based input materials may be assigned to such chemical products.

The chemical production network may include one or more production chain(s) that further process or chemically convert the cracker intermediates. The cracker intermediates may be further processed or chemically converted by one or more production chain(s) inside the chemical production network. The cracker intermediates may be processed to downstream products. The downstream products may be provided to the exit point of the chemical production network. The downstream products may hence be chemical products. The environmental attributes of the recycled or bio-based input materials may be assigned to such chemical products. The cracker intermediates may be processed via one or more downstream intermediates to chemical products. The environmental attributes of the recycled or bio-based input materials may be assigned to such chemical products. Chemical products may include polymers, specialty chemicals, consumer chemicals, solvents, pharmaceuticals, or the like.

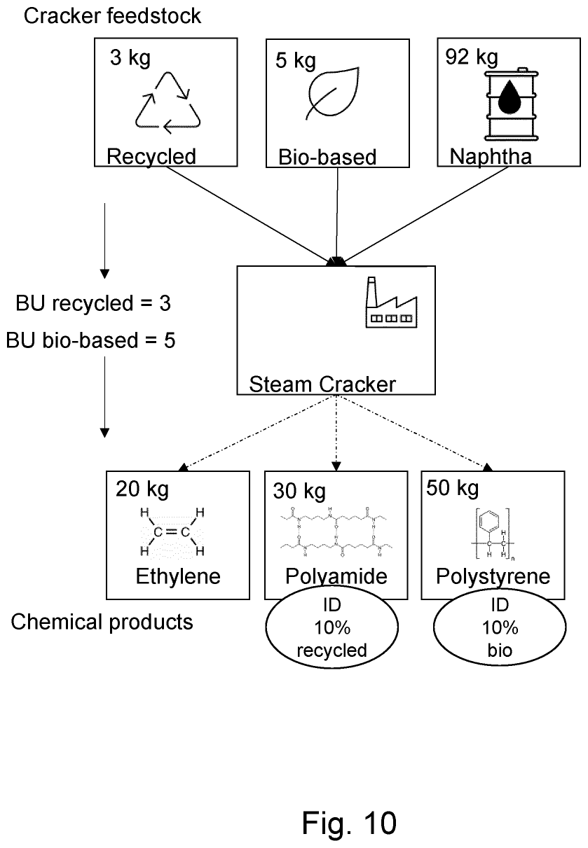

For illustration purposes and to further explain the methods for allocating environmental attributes to virtual balancing accounts and assigning environmental attributes to chemical products as described in the context of FIGS. 6-8, FIG. 10 illustrates examples of the conversion to balancing units and assignment to chemical products.

Based on the non-limiting example of FIG. 9 bio-based and recycled feedstock may be provided to the steam cracker. For illustrative purposes the following input materials may be provided to the steam cracker in the following amounts:
3 kg recycled input material such as pyrolysis oil produced from mixed plastics waste,
5 kg bio-based input material such as bio-naphtha from vegetable oil that is kosher and vegan,
92 kg of naphtha.

On providing these input materials to the steam cracker, the input materials enter the chemical production network. The environmental attributes of the recycled and bio-based input materials may be provided to the computer interface configured to convert such attributes to balancing units and to allocate the balancing units to respective virtual balancing accounts. In the simplest example, the balancing units (BUs) may correspond to the amounts of the respective materials. In the example, the environmental attribute pyrolysis oil from recycled mixed plastics waste may correspond to 3 BUs and the environmental attribute bio-naphtha from bio-based food waste may correspond to 5 BUs. In this example a simplified weight-based approach is used for illustrative purposes only. Other approaches may be based on energy, atom counting such as carbon atoms, molecule counting such as methane, or include losses that occur in production.

In more elaborate embodiments the balancing units may be determined based on a more complex conversion factor taking chemical and/or physical differences between input materials and their associated yield into account. The conversion factor may quantify the differences in chemical and/or physical properties of replacing fossil input material(s) by non-fossil input material(s). The conversion factor may relate the use of conventional input material(s) to the use of input material(s) associated with one or more environmental attribute(s). The conversion factor may depend on carbon atoms, methane molecules, energy properties, process properties or any other suitable factors for quantifying the environmental impact of the environmental attribute. For instance, the lower or higher heating value (LHV, HHV) of the fossil and the non-fossil input material may be taken into account. Further for instance, material losses that occur in the processing of the fossil or the non-fossil input material may be taken into account. Further for instance, exempted steam cracker products, intermediates or production chains may be taken into account. Further for instance, only pre-selected production chains may be taken into account. This way the environmental impact of the non-fossil input materials may be quantified with reference to fossil input materials.

The steam cracker may produce cracker products, which may be further processed and chemically converted. In the illustrative example, 20 kg ethylene as cracker product, 30 kg polyamide and 50 kg polystyrene may be provided to the exit point of the chemical production network. Since for the production of such products 3 kg recycled and 5 kg bio-based input materials were used, the environmental attributes allocated in BUs to the respective balancing accounts can be assigned to such products. For instance, 3 BUs recycled may be assigned to Polyamide, which corresponds to 10% recycled content, and 5 BUs bio-based may be assigned to Polystyrene, which corresponds to 10% bio-based content.

For illustration purposes and to further explain the methods for allocating environmental attributes to virtual balancing accounts as described in the context of FIGS. 6-8, FIGS. 11a-c illustrate examples of balancing accounts to manage the allocation and assignment of balancing units.

Based on the non-limiting example of FIG. 9 bio-based and recycled feedstock may be provided to the steam cracker. Material data related to the bio-based and recycled feedstock and the respective environmental attributes may be provided to the computing interface configured to convert such attributes to balancing units and to allocate the balancing units to respective virtual balancing accounts. In the simplest example, the environmental attribute pyrolysis oil from recycled mixed plastics waste may correspond to 3 BUs and the environmental attribute bio-naphtha from bio-based vegetable oil may correspond to 5 BUs. The so determined BUs may further be associated with metadata signifying the respective environmental attributes as recycled, pyrolysis oil and recycled tires as well as bio-based, bio-naphtha, vegetable oil.

For allocation the virtual balancing system may be provided. The virtual balancing system may include balancing accounts associated with metadata relating to environmental attribute, input material type, waste stream type, biomass type, and balancing units. As for example shown in FIG. 11a, the balancing accounts may be associated with meta data relating to environmental attribute type recycled or bio-based, input material type pyrolysis oil, bio-naphtha or bio-gas, input material origins tires, mixed plastics waste, vegetable oil or food waste, respectively. Furthermore, the balancing accounts may be associated with an account balance quantifying the BUs.

For allocation of the 3 BUs and the 5 BUs the metadata of the balancing accounts may be matched with the metadata associated with such BUs. The BU metadata may be provided from the input material data. Once a match in metadata is found the respective BUs are allocated to the matched account. Hence in the example, the 3 BUs pyrolysis oil from recycled mixed plastics waste may be allocated to account BU recycled 2 and the 5 BUs bio-naphtha from bio-based vegetable oil may be allocated to account BU bio 1.

The virtual balancing system may be based on balancing accounts with static or dynamic meta data. For instance, if metadata provided via the environmental attribute associated with the input material does not correspond to any metadata of balancing accounts provided, a new account associated with such metadata may be provided. Alternatively, or additionally, the environmental attribute may be allocated to the account associated with the greatest match in metadata. Here greatest may refer to the maximal number of matching meta data points, in particular account metadata points matching at least in part with environmental attribute metadata points. For example, the environmental attribute may provide more metadata than any balancing account. In such a scenario the account with metadata points matching at least in part with metadata points of the environmental attribute may be selected for allocation.

The metadata structure shown in FIG. 11a is only one example. FIG. 11b illustrates another example. Here a hierarchical tree of metadata (leafs) with different levels may be associated to the balancing accounts. For example, the top level may be bio-based or recycled, the next level may specify the input material type for recycled or bio-based, respectively, and so on.

The metadata structures shown in FIGS. 11a,b is only one examples. FIG. 11c illustrates another example. In this example the virtual accounting system may be associated with metadata relating to the environmental attribute recycled and the input material type pyrolysis oil. The virtual accounting system may include two balancing accounts, BU1 and BU2. BU1 may be associated with waste stream type tires and the balancing units allocated to BU1. BU2 may be associated with waste stream type mixed plastics and the balancing units allocated to BU2.

FIG. 11a-c are only examples based on metadata attached to the virtual accounting system or the balancing accounts. Other options to realize such a data structure exist. For example, the virtual balancing system and the balancing accounts may be fully abstracted and each BU entering the system may have its own metadata.

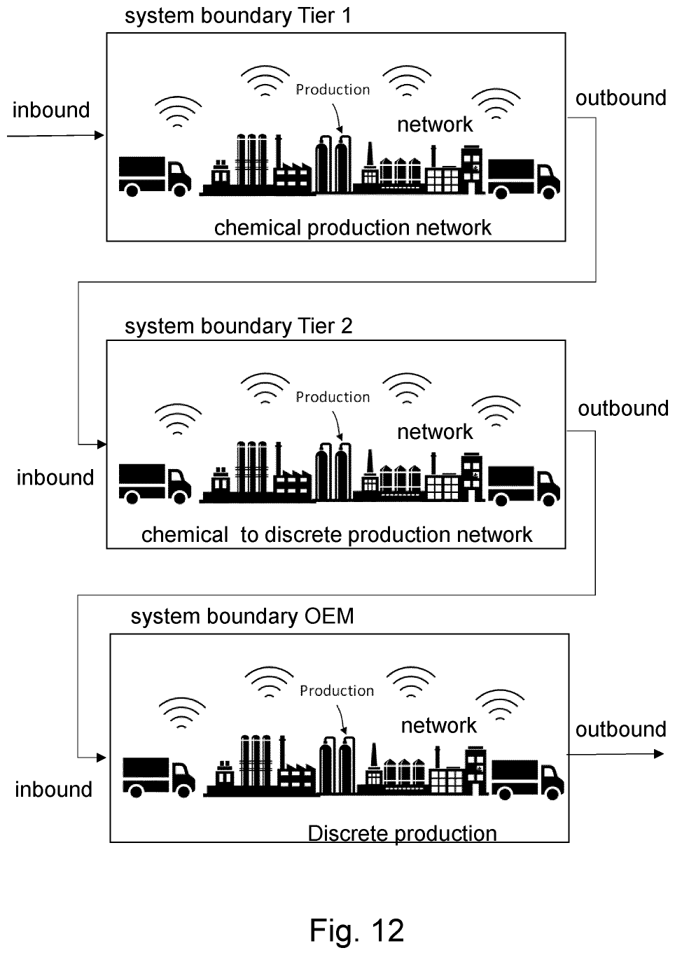

FIG. 12 illustrates an example of a method for producing at least one chemical product associated with at least one environmental attribute for a supply chain.

The example illustrates two tiers and an original equipment manufacturer. Tier 1 may be a chemical producer operating the chemical production network. The chemical production network may be associated with a system boundary. The system boundary may signify the physical boundary of the chemical production network. The input material(s) entering the chemical production network at any stage of the production network, or the system boundary of the chemical production network may signify entry points to the chemical production network. On entry of input material(s) associated with one or more environmental attributes the environmental attribute(s) may be decoupled from the physical material flow of the input material(s) through the chemical production network. Such separation of the physical input material and the virtual environmental attribute may be provided by the method for registering at least one input material associated with at least one environmental attribute as described herein and as for example illustrated in FIG. 5a (inbound). Chemical product(s) may be produced from input material(s) by the chemical production network. At least one chemical product may be produced by the chemical production network and the chemical product may be linked to at least one environmental attribute as described herein and illustrated in FIG. 5b (outbound).

The chemical product(s) associated with at least one environmental attribute as produced by the chemical production network may be provided to the next tier. In the illustrated example tier 2 may be a production network producing discrete products from chemical product(s) or chemical products. Discrete products may be any products associated with distinct physical units. Discrete manufacturing in contrast to process manufacturing use such discrete products to assemble other discrete products. Chemical production in contrast uses process manufacturing where input material(s) are mixed and chemically converted to chemical output product(s). Such output product(s) are chemical product(s) that may be transferred in discrete units, such as containers or wreath. The chemical product(s) are such are however no discrete product(s). The chemical to discrete production network may be associated with a system boundary as described above. Similar to the chemical production network the chemical to discrete production network may on inbound register input material(s) associated with at least one environmental attribute, which may be the chemical product(s) of the chemical production network as described herein or illustrated in FIG. 5a (inbound). Discrete output product(s) may be produced by the chemical to discrete production network. At least one discrete output product may be linked to at least one environmental attribute (outbound).

The discrete product(s) associated with at least one environmental attribute as produced by the chemical to discrete production network may be provided to an original equipment manufacturer producing end products. As described herein and in the context of FIGS. 5a and 6, the discrete product(s) and associated environmental attribute(s) may be decoupled on registration (inbound). As described herein and illustrated in FIGS. 5b and 7 after decoupling the environmental attribute may be linked to the end product as described herein and illustrated in FIG. 5b (outbound).

The one or more environmental attribute(s) assigned to the chemical product identifier may be used to provide at least one discrete product identifier relating at least one discrete product or at least one end product identifier specifying at least one end product of a product supply chain to the one or more environmental attribute(s). The chemical product associated with one or more environmental attribute(s) may be used to produce at least one discrete product or at least one end product of a product supply chain associated with the one or more environmental attribute(s).

This way the environmental attributes associated with any input material(s) may be tracked through the value chain up to the end product. The methods, apparatuses and systems described herein enable transparency from early stages of the value chain on chemical product level to end stage of the value chain on end product level. Through linking of physical material and environmental attribute environmentally friendly products and more sustainable production can be made transparent and tracked.

1.-16. (canceled)

16. A method for producing at least one chemical product associated with one or more environmental attributes, wherein the chemical product is produced by a chemical production network, the method comprising:
providing one or more input material(s) associated with one or more environmental attribute(s) to the chemical production network,
providing material data related to the one or more input material(s) and respective environmental attributes to a computing interface,
allocating the one or more environmental attribute(s) to a virtual balancing system, wherein the virtual balancing system includes balancing accounts associated with one or more environmental attribute(s),
producing at least one chemical product,
assigning at least one environmental attribute from at least one balancing account associated with the respective environmental attribute to at least one chemical product.

17. The method of claim 16, wherein the environmental attribute(s) relate(s) to a renewable, a bio-based and/or a recycled content.

18. The method of claim 16, wherein the virtual balancing system or the balancing account is associated with one or more characteristic(s) of the input material.

19. The method of claim 16, wherein the virtual balancing system or the balancing account is associated with an input material type, a waste stream type, a biomass type, a renewable type, an allocation scheme, or combinations thereof.

20. The method of claim 16, wherein the virtual balancing system or the balancing account is associated with one or more input material type(s) including pyrolysis oil, pyrolysis gas, synthesis gas, hydrogen, naphtha, methane, ethane, propane, chemicals, or combinations thereof.

21. The method of claim 16, wherein the virtual balancing system or the balancing account is associated with one or more waste stream type(s) linked to at least the environmental attribute recycled.

22. The method of claim 16, wherein the virtual balancing system or the balancing account is associated with one or more biomass type(s) linked to at least the environmental attribute bio-based or renewable

23. The method of claim 16, wherein the virtual balancing system or the balancing account is associated with metadata specifying the relationship between the input material(s) associated with one or more environmental attribute(s) and the chemical product.

24. The method of claim 22, wherein the relationship relates to the chemical production network, one or more production chain(s), one or more chemical product(s), one or more chemical product classes or combinations thereof.

25. The method of claim 16, wherein at least one target environmental attribute for the chemical product is provided and based on the at least one target environmental attribute at least one balancing account for deallocation the respective environmental attribute is selected.

26. The method of claim 16, wherein assigning the at least one environmental attribute to the at least one chemical product includes providing at least one target environmental attribute for the chemical product,
checking the environmental attribute balance of the balancing account associated with the at least one target environmental attribute and assigning the at least one environmental attribute from the respective balancing account to the at least one chemical product, if the balance is sufficient, and/or
checking the respective balancing accounts are associated with input material(s) used in the production chain of the chemical product and assigning the at least one environmental attribute from the respective balancing account to the at least one chemical product if the respective balancing account is associated with input material(s) used in the production chain of the chemical product.

27. The method of claim 16, wherein assigning at least one environmental attribute to the at least one chemical product includes providing a chemical product identifier associated with the produced chemical product and assigning the environmental attribute from the balancing account to the chemical product identifier.

28. A computer-implemented method for assigning at least one environmental attribute to at least one chemical product produced by a chemical production network, the method comprising:
providing access to a virtual balancing system, wherein the virtual balancing system includes more than one balancing account associated with at least one environmental attribute and an environmental attribute balance with environmental attribute(s) allocated from one or more input material(s) provided to the chemical production network,
based on one or more environmental attribute(s) to be assigned to the at least one chemical product selecting at least one balancing account from the virtual balancing system,
assigning at least one environmental attribute from at least one balancing account associated with the respective environmental attribute to at least one chemical product.

29. An apparatus for assigning at least one environmental attribute to at least one chemical product produced by a chemical production network, the method comprising:
an account provider configured to provide access to a virtual balancing system, wherein the virtual balancing system includes more than one balancing account associated with at least one environmental attribute and an environmental attribute balance with environmental attribute(s) allocated from one or more input material(s) provided to the chemical production network,
an outbound assignor configured to select at least one balancing account from the virtual balancing system based on or more environmental attribute(s) to be assigned to the at least one chemical product and configured to assign the at least one environmental attribute from at least one balancing account associated with the respective environmental attribute to at least one chemical product.

30. A system for producing at least one chemical product associated with one or more environmental attributes, wherein the chemical product is produced by a chemical production network including one or more production chain(s), the system comprising:
a chemical production network configured to produce at least one chemical product based on one or more input material(s) provided to the chemical production network, wherein the one or more input material(s) are associated with one or more environmental attribute(s);
an inbound allocator configured to provide material data related to one or more input material(s) and respective environmental attributes to a computing interface and to allocate one or more environmental attribute(s) to a virtual balancing system, wherein the virtual balancing system includes balancing accounts associated with one or more environmental attribute(s),
an outbound assignor configured to assign at least one environmental attribute from at least one balancing account associated with the respective environmental attribute to at least one chemical product.

31. Use of the chemical product associated with one or more environmental attribute(s) as provided by any of the methods of claim 16 to produce at least one discrete product or at least one end product of a product supply chain associated with the one or more environmental attribute(s).
